(19) United States
(12) Patent Application Publication
(10) Pub. No.: US 2024/0131004 A1
(43) Pub. Date: Apr. 25, 2024

(54) FUNCTIONALIZED 1,3-BENZENE DIOLS AND THEIR METHOD OF USE FOR THE TREATMENT OF INFLAMMATION AND PAIN

(71) Applicant: KANNALIFE SCIENCES, INC., Doylestown, PA (US)

(72) Inventors: Douglas BRENNEMAN, North Wales, PA (US); William A. KINNEY, Newtown, PA (US); Mark E. MCDONNELL, Lansdale, PA (US); Dean PETKANAS, Lloyd Harbor, NY (US)

(21) Appl. No.: 18/275,159

(22) PCT Filed: Jan. 31, 2022
(86) PCT No.: PCT/US2022/014557

§ 371 Date: Jul. 31, 2023

Related U.S. Application Data
(60) Provisional application No. 63/144,471, filed on Feb. 1, 2021.

(51) Int. Cl. A61K 31/397; A61P 29/00

(52) U.S. Cl. CPC A61K 31/397; A61P 29/00; A61K 31/475

(57) ABSTRACT

A method of treating or preventing inflammation and pain includes administering to a subject a functionalized 1,3-benzene diol represented by a formula selected from formulas (I)-(X) as defined herein, and hydrates, solvates, pharmaceutically acceptable salts, and complexes thereof. Further disclosed is a chemotherapeutic method, including administering to a patient: (1) at least one chemotherapeutic agent and (2) at least one functionalized 1,3-benzene diol represented by a formula selected from formulas (I)-(X) as defined herein, and hydrates, solvates, pharmaceutically acceptable salts, and complexes thereof, which is effective to treat or prevent inflammation and pain associated with administering the at least one chemotherapeutic agent. A chemotherapeutic composition includes a chemotherapeutic agent selected from the group consisting of vincristine, vinblastine, paclitaxel, docetaxel, bortezomib, cisplatin, oxaliplatin and carboplatin, and a functionalized 1,3-benzene diol represented by a formula selected from formulas (I)-(X) as defined herein, and hydrates, solvates, pharmaceutically acceptable salts, and complexes thereof.

### CROSS-REFERENCE TO RELATED APPLICATIONS

This PCT application claims the benefit under 35 U.S.C. § 119(e) of U.S. Application No. 63/144,471 filed on Feb. 1, 2021 entitled “FUNCTIONALIZED 1,3-BENZENE DIOLS AND THEIR METHOD OF USE FOR THE TREATMENT OF INFLAMMATION AND PAIN” and whose entire disclosure is incorporated by reference herein.

### FIELD OF INVENTION

This invention relates to methods and compositions for the treatment of inflammation and pain, and more particularly to the use of functionalized 1,3-benzene diols in such methods and compositions.

### BACKGROUND OF THE INVENTION

Inflammation is a multi-organ response to injury and infection that can result in chronic pain. Neuroinflammation is a localized inflammation in the peripheral nervous system (PNS) and central nervous system (CNS). A salient feature of neuroinflammation is the activation of multiple cell types in dorsal root ganglion (DRG), spinal cord and brain which leads to the production of proinflammatory cytokines and chemokines that can evoke acute and chronic pain. See, e.g., Ji et al., 2018.

Neuropathic pain is a pathophysiologic condition produced by damage to, or pathologic changes in, the peripheral and central nervous systems that has an inflammatory component (Matsuda et al., 2019). It is characterized by abnormal pain sensations, including spontaneous pain, hyperalgesia (i.e., increased sensitivity to a typically noxious stimulus) and allodynia (i.e., increased sensitivity to a typically non-noxious stimulus) that typically lack an apparent physiologic function.

Neuropathic pain remains a challenging neurologic disorder that adversely affects quality of life and presents a large unmet medical need for improved therapies. Current treatments for neuropathic pain are few and of limited effectiveness. The three major classes of compounds most often used are tricyclic antidepressants (TCA's), anticonvulsants (AED's), and, to a lesser extent, mu-opioid receptor agonists. Clinical data indicate that approximately 50% of treated individuals are unresponsive to current pharmacotherapies, and in those that receive some benefit, pain relief is typically incomplete (Bonezzi and Demartini 1999).

Chemotherapy-Induced Peripheral Neuropathy (CIPN) is a progressive, enduring, and often irreversible condition featuring pain, numbness, tingling and sensitivity to cold in the hands and feet (sometimes progressing to the arms and legs) that afflicts between 30% and 40% of patients undergoing chemotherapy (Gutierrez-Gutierrez et al 2010). Chemotherapy drugs associated with CIPN include the vinca alkaloids vincristine and vinblastine, the taxanes paclitaxel and docetaxel, the proteasome inhibitors such as bortezomib, and the platinum-based drugs cisplatin, oxaliplatin and carboplatin. Three key mechanisms involved in the development of CIPN are mitochondrial dysfunction, loss of Ca⁺⁺ homeostasis, and oxidative stress (Han et al. 2013). Associated effects on peripheral nerves can lead to peripheral and central nitroxidative stress and inflammation, sensitization and spontaneous activity of peripheral nerve fibers, and hyperexcitability in the dorsal column of the spinal cord, leading to ascending pain pathway sensitization.

Accordingly, it is desired to provide new compositions and methods useful for treating one or more of neuroinflammation, neuropathic pain and CIPN.

### BRIEF SUMMARY OF THE INVENTION

Accordingly, a first aspect of the invention is a method of treating or preventing inflammation and pain in a subject, said method comprising administering to the subject an effective amount of a functionalized 1,3-benzene diol represented by a formula selected from the group consisting of:
(a) formula (I):

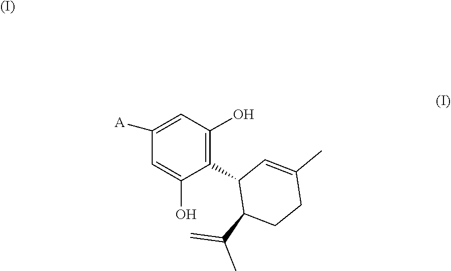

including hydrates, solvates, pharmaceutically acceptable salts, prodrugs and complexes thereof, wherein:
A is selected from the group consisting of

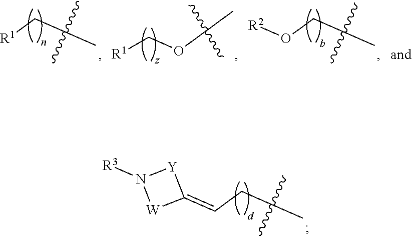

z is 0, 1, or 2;
when A is

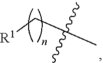

R¹ is selected from the group consisting of

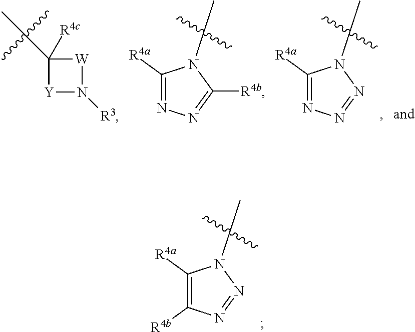

when A is

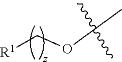

and z is 0, R¹ is

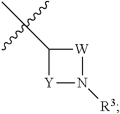

when A is

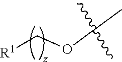

and z is 1, R¹ is

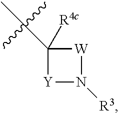

when A is

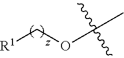

and z is 2, R¹ is selected from the group consisting of

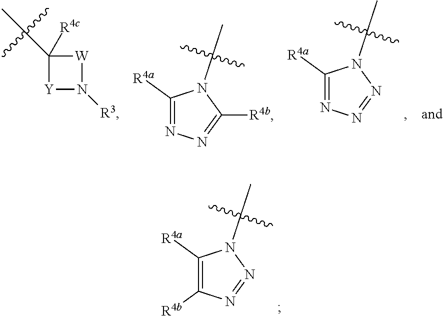

when R¹ is

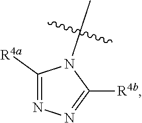

n is not 0;
when R¹ is

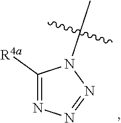

n is not 0;
when R¹ is

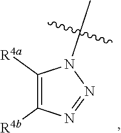

n is not 0;
R² is

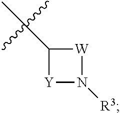

W is (CH₂)ₘ;
m is 1 or 2;
Y is (CH₂)_{q};
q is 1 or 2;
n is 0, 1, 2, or 3;
b is 0, 1, 2, or 3;
d is 0, 1, 2, or 3;
R³ is selected from the group consisting of COR⁵, CO₂R⁶, CONR^{7a}R^{7b}, SO₂NR^{7a}R^{7b}, SO₂R⁸, and optionally substituted heteroaryl;
R^{4a} and R^{4b} are each independently selected from the group consisting of hydrogen and C₁₋₆ alkyl;
R^{4c} is selected from the group consisting of hydrogen and OH;
R⁵ is selected from the group consisting of hydrogen, unsubstituted C₁₋₆ alkyl, substituted C₁₋₆ alkyl, unsubstituted heteroaryl, substituted heteroaryl, —C(R^{9a}R^{9b})NR^{7a}R^{7b}, and —C(R^{9a}R^{9b})OR¹⁰,
R⁶ is unsubstituted C₁₋₆ alkyl or substituted C₁₋₆ alkyl;
R^{7a} and R^{7b} are each independently selected from the group consisting of hydrogen, unsubstituted C₁₋₆ alkyl and substituted C₁₋₆ alkyl;
R⁸ is selected from the group consisting of hydrogen, unsubstituted C₁₋₆ alkyl, substituted C₁₋₆ alkyl, unsubstituted heteroaryl and substituted heteroaryl;
R^{9a} and R^{9b} are each independently selected from the group consisting of hydrogen, C₁₋₆ alkyl, C₃₋₇ branched alkyl, CH₂OH, CH(OH)CH₃, CH₂Ph, CH₂(4-OH-Ph), (CH₂)₄NH₂, (CH₂)₃NHC(NH₂)NH, CH₂(3-indole), CH₂(5-imidazole), CH₂CO₂H, CH₂CH₂CO₂H, CH₂CONH₂, and CH₂CH₂CONH₂; and
R¹⁰ is selected from the group consisting of hydrogen and C₁₋₆ alkyl;
(b) formula (II):

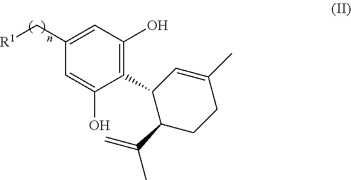

including hydrates, solvates, pharmaceutically acceptable salts, and complexes thereof, wherein R¹ and n of formula (II) are as defined above with respect to formula (I);
(c) formula (III):

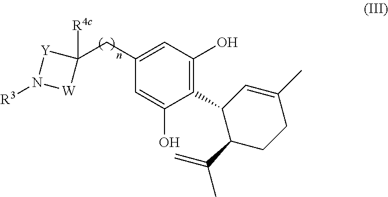

including hydrates, solvates, pharmaceutically acceptable salts, and complexes thereof, wherein R³, R^{4c}, Y, W, and n of formula (III) are as defined above with respect to formula (I);
(d) formula (IV):

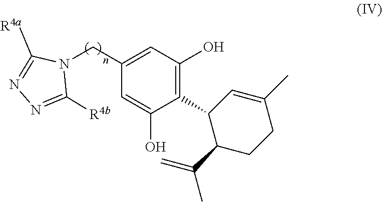

including hydrates, solvates, pharmaceutically acceptable salts, and complexes thereof, wherein n, R^{4a} and R^{4b} are as defined above with respect to formula (I);
(e) formula (V):

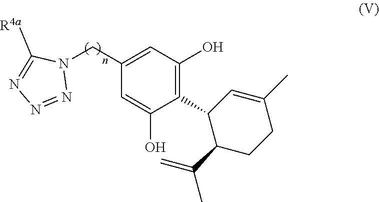

including hydrates, solvates, pharmaceutically acceptable salts, and complexes thereof, wherein n and R^{4a} are as defined above with respect to formula (I);
(f) formula (VI):

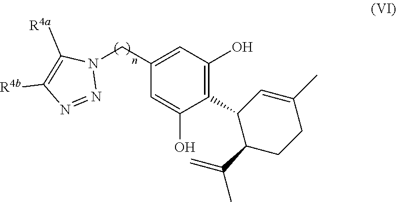

including hydrates, solvates, pharmaceutically acceptable salts, and complexes thereof, wherein n, R^{4a} and R^{4b} are as defined above with respect to formula (I);
(g) formula (VII):

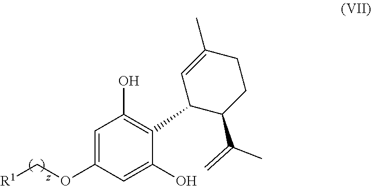

including hydrates, solvates, pharmaceutically acceptable salts, and complexes thereof, wherein R¹ and z are as defined above with respect to formula (I);
(h) formula (VIII):

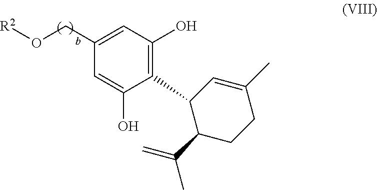

including hydrates, solvates, pharmaceutically acceptable salts, and complexes thereof, wherein R² and b are as defined above with respect to formula (I);
(i) formula (IX):

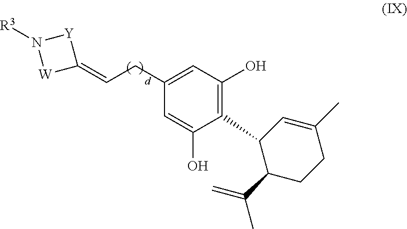

including hydrates, solvates, pharmaceutically acceptable salts, and complexes thereof, wherein R³, Y, W, and d of formula (IX) are as defined above with respect to formula (I); and
(j) formula (X):

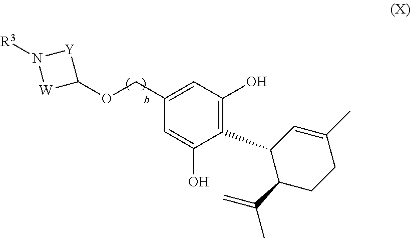

including hydrates, solvates, pharmaceutically acceptable salts, and complexes thereof, wherein R³, Y, W, and b of formula (X) are as defined above with respect to formula (I).

In certain embodiments of the inventive method, the functionalized 1,3-benzene diol is represented by the following formula:

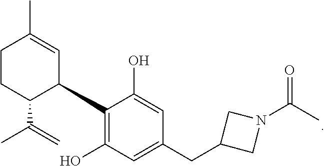 [SMILES: C=C(C)[C@@H]1CCC(C)=C[C@H]1c1c(O)cc(CC2CN(C(C)=O)C2)cc1O]

In certain embodiments of the inventive method, the inflammation and pain are associated with a disorder in which GPR55 receptor is expressed in a target tissue of the subject and the functionalized 1,3-benzene diol is a GPR55 antagonist.

In certain embodiments of the inventive method, the disorder is selected from the group consisting of a chemotherapy-induced peripheral neuropathy, a traumatic brain injury, a traumatic spinal cord injury, a stroke, an autoimmune disease, a viral infection, a surgical trauma, osteoarthritis and chronic opioid treatment.

In certain embodiments of the inventive method, the disorder is a chemotherapy-induced peripheral neuropathy and the functionalized 1,3-benzene diol is administered before, during and/or after administering to the subject a chemotherapeutically effective amount of at least one chemotherapeutic agent selected from the group consisting of vincristine, vinblastine, paclitaxel, docetaxel, bortezomib, cisplatin, oxaliplatin and carboplatin.

In certain embodiments of the inventive method, the functionalized 1,3-benzene diol is effective to decrease levels of circulating or tissue cytokines produced in response to the administering of the at least one chemotherapeutic agent.

In certain embodiments of the inventive method, the disorder is chronic opioid treatment, and the functionalized 1,3-benzene diol decreases CNS inflammation associated with chronic opioid use and/or reduces or eliminates opioid dependency.

In certain embodiments of the inventive method, the disorder is diabetic neuropathy.

In certain embodiments of the inventive method, the disorder is post-traumatic stress disorder.

In certain embodiments of the inventive method, the disorder is a neurodegenerative disease that has an oxidative stress component.

In certain embodiments of the inventive method, the subject is a human.

A second aspect of the invention is a chemotherapeutic method, comprising:
administering to a patient a chemotherapeutically effective amount of at least one chemotherapeutic agent selected from the group consisting of vincristine, vinblastine, paclitaxel, docetaxel, bortezomib, cisplatin, oxaliplatin and carboplatin; and
administering to the patient at least one functionalized 1,3-benzene diol in an amount effective to treat or prevent inflammation and pain associated with administering the at least one chemotherapeutic agent to the patient, wherein the at least one functionalized 1,3-benzene diol is represented by a formula selected from the group consisting of formula (I), formula (II), formula (III), formula (IV), formula (V), formula (VI), formula (VII), formula (VIII), formula (IX), and formula (X) as defined above, and hydrates, solvates, pharmaceutically acceptable salts, and complexes thereof

In certain embodiments of the inventive chemotherapeutic method, the functionalized 1,3-benzene diol is represented by the following formula:

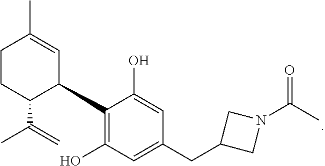 [SMILES: C=C(C)[C@@H]1CCC(C)=C[C@H]1c1c(O)cc(CC2CN(C(C)=O)C2)cc1O]

In certain embodiments of the inventive chemotherapeutic method, the functionalized 1,3-benzene diol and the chemotherapeutic agent are administered together.

A third aspect of the invention is a chemotherapeutic composition, comprising:
a chemotherapeutic agent selected from the group consisting of vincristine, vinblastine, paclitaxel, docetaxel, bortezomib, cisplatin, oxaliplatin and carboplatin; and
a functionalized 1,3-benzene diol represented by a formula selected from the group consisting of formula (I), formula (II), formula (III), formula (IV), formula (V), formula (VI), formula (VII), formula (VIII), formula (IX), and formula (X) as defined above, and hydrates, solvates, pharmaceutically acceptable salts, and complexes thereof.

In certain embodiments of the chemotherapeutic composition, the chemotherapeutic agent is provided in a chemotherapeutically effective amount and the at least one functionalized 1,3-benzene diol is provided in an amount effective to treat or prevent inflammation and pain associated with administering the at least one chemotherapeutic agent to the patient.

In certain embodiments of the chemotherapeutic composition, the functionalized 1,3-benzene diol is represented by the following formula:

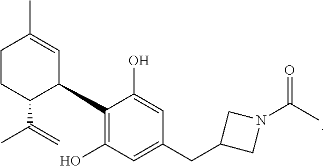 [SMILES: C=C(C)[C@@H]1CCC(C)=C[C@H]1c1c(O)cc(CC2CN(C(C)=O)C2)cc1O]

### BRIEF DESCRIPTION OF THE DRAWINGS

The invention will be described in conjunction with the following drawings, wherein:

FIG. 1: shows cellular viability in DRG cultures as measured with Alamar Blue after a reversal treatment paradigm: hours 0-24 with 3 μM paclitaxel followed by hours 8-24 treatment with various concentrations of KLS-13019.

FIG. 2: shows GPR55 immunoreactive area present in cell bodies of DRG neurons as measured with high content fluorescence microimaging after a reversal treatment paradigm: hours 0-24 with 3 μM paclitaxel followed by hours 8-24 treatment with various concentrations of KLS -13019.

FIG. 3: shows interleukin-6 immunoreactive area present in neurites and cell bodies of DRG neurons as measured with high content fluorescence microimaging after a reversal treatment paradigm: hours 0-24 with 3 μM paclitaxel followed by hours 8-24 treatment with various concentrations of KLS-13019.

FIG. 4: shows interleukin-1β immunoreactive area present in neurites and cell bodies of DRG neurons as measured with high content fluorescence microimaging after a reversal treatment paradigm: hours 0-24 with 3 μM paclitaxel followed by hours 8-24 treatment with various concentrations of KLS-13019.

FIG. 5: shows a time course of LPIA-mediated effects on neuritic interleukin-1β, interleukin-6 and GPR55 immunoreactive areas in DRG neurons.

FIG. 6: shows cellular viability in hippocampal cultures as measured with Alamar Blue after a reversal treatment paradigm: hours 0-8 with 1 nM LPIA followed by hours 4-8 treatment with various concentrations of KLS-13019.

FIG. 7: shows GPR55 immunoreactive area present in neurites of hippocampal neurons as measured with high content fluorescence microimaging in DRG cultures after 6 hours treatment with 1 nM LPIA and co-treatment with various concentrations of KLS-13019.

FIG. 8: shows KLS-13019-mediated antagonistic inhibition of β-arrestin-2 levels after stimulation of human GPR55 with lysophosphatidylinositol in DiscoverX cells.

FIG. 9: shows NLRP3 immunoreactive area present in neurites of hippocampal neurons as measured with high content fluorescence microimaging after a reversal treatment paradigm: hours 0-8 with 1 nM LPIA followed by hours 5-8 treatment with various concentrations of KLS -13019.

### DETAILED DESCRIPTION OF PREFERRED EMBODIMENTS OF THE INVENTION

### Glossary

As used herein, the term “halogen” shall mean chlorine, bromine, fluorine and iodine.

As used herein, unless otherwise noted, “alkyl” and/or “aliphatic” whether used alone or as part of a substituent group refers to straight and branched carbon chains having 1 to 20 carbon atoms or any number within this range, for example 1 to 6 carbon atoms or 1 to 4 carbon atoms. Designated numbers of carbon atoms (e.g. C₁₋₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tert-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁₋₆alkyl)₂amino, the alkyl groups may be the same or different.

As used herein, the terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups can be optionally substituted. Nonlimiting examples of alkenyl groups include ethenyl, 3-propenyl, 1-propenyl (also 2-methylethenyl), isopropenyl (also 2-methylethen-2-yl), buten-4-yl, and the like. Nonlimiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Nonlimiting examples of alkynyl groups include ethynyl, prop-2-ynyl (also propargyl), propyn-1-yl, and 2-methyl-hex-4-yn-1-yl. Nonlimiting examples of substituted alkynyl groups include, 5-hydroxy-5-methylhex-3-ynyl, 6-hydroxy-6-methylhept-3-yn-2-yl, 5-hydroxy-5-ethylhept-3-ynyl, and the like.

As used herein, “cycloalkyl,” whether used alone or as part of another group, refers to a non-aromatic carbon-containing ring including cyclized alkyl, alkenyl, and alkynyl groups, e.g., having from 3 to 14 ring carbon atoms, preferably from 3 to 7 or 3 to 6 ring carbon atoms, or even 3 to 4 ring carbon atoms, and optionally containing one or more (e.g., 1, 2, or 3) double or triple bond. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Cycloalkyl rings can be optionally substituted. Nonlimiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

“Haloalkyl” is intended to include both branched and straight-chain saturated aliphatic hydrocarbon groups having the specified number of carbon atoms, substituted with 1 or more halogen. Haloalkyl groups include perhaloalkyl groups, wherein all hydrogens of an alkyl group have been replaced with halogens (e.g., —CF₃, —CF₂CF₃). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

The term “alkoxy” refers to the group —O-alkyl, wherein the alkyl group is as defined above. Alkoxy groups optionally may be substituted. The term C₃-C₆ cyclic alkoxy refers to a ring containing 3 to 6 carbon atoms and at least one oxygen atom (e.g., tetrahydrofuran, tetrahydro-2H-pyran). C₃-C₆ cyclic alkoxy groups optionally may be substituted.

The term “aryl,” wherein used alone or as part of another group, is defined herein as an unsaturated, aromatic monocyclic ring of 6 carbon members or to an unsaturated, aromatic polycyclic ring of from 10 to 14 carbon members. Aryl rings can be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

The term “arylalkyl” and “aralkyl” refer to the group —alkyl-aryl, where the alkyl and aryl groups are as defined herein. Aralkyl groups of the invention are optionally substituted. Examples of arylalkyl groups include, for example, benzyl, 1-phenylethyl, 2-phenylethyl, 3-phenylpropyl, 2-phenylpropyl, fluorenylmethyl and the like.

The terms “heterocyclic” and “heterocycle” and “heterocylyl,” whether used alone or as part of another group, are defined herein as one or more ring having from 3 to 20 atoms wherein at least one atom in at least one ring is a heteroatom selected from nitrogen (N), oxygen (O), or sulfur (S), and wherein further the ring that includes the heteroatom is non-aromatic. In heterocycle groups that include 2 or more fused rings, the non-heteroatom bearing ring may be aryl (e.g., indolinyl, tetrahydroquinolinyl, chromanyl). Exemplary heterocycle groups have from 3 to 14 ring atoms of which from 1 to 5 are heteroatoms independently selected from nitrogen (N), oxygen (O), or sulfur (S). One or more N or S atoms in a heterocycle group can be oxidized. Heterocycle groups can be optionally substituted.

Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro -1H-pyrrolizinyl, 3a,4,5,6,7,7a-hexahydro-1H-benzo[d]imidazolyl, 3a,4,5,6,7,7a -hexahydro-1H-indolyl, 1,2,3,4-tetrahydroquinolinyl, chromanyl, isochromanyl, indolinyl, isoindolinyl, and decahydro-1H-cycloocta[b]pyrrolyl.

The term “heteroaryl,” whether used alone or as part of another group, is defined herein as one or more rings having from 5 to 20 atoms wherein at least one atom in at least one ring is a heteroatom chosen from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl (e.g., benzofuranyl, benzothiophenyl, indolyl). Exemplary heteroaryl groups have from 5 to 14 ring atoms and contain from 1 to 5 ring heteroatoms independently selected from nitrogen (N), oxygen (O), or sulfur (S). One or more N or S atoms in a heteroaryl group can be oxidized. Heteroaryl groups can be substituted. Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, and isoquinolinyl.

One non-limiting example of a heteroaryl group as described above is C₁-C₅ heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

Unless otherwise noted, when two substituents are taken together to form a ring having a specified number of ring atoms (e.g., R² and R³ taken together with the nitrogen (N) to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

For the purposed of the invention fused ring units, as well as spirocyclic rings, bicyclic rings and the like, which comprise a single heteroatom will be considered to belong to the cyclic family corresponding to the heteroatom containing ring. For example, 1,2,3,4-tetrahydroquinoline having the formula:

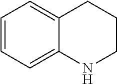

is, for the purposes of the invention, considered a heterocyclic unit. 6,7-Dihydro-5H-cyclopentapyrimidine having the formula:

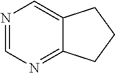

is, for the purposes of the invention, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

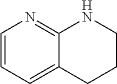

is, for the purposes of the invention, considered a heteroaryl unit.

Whenever a term or either of their prefix roots appear in a name of a substituent, the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is used throughout the specification. The term “substituted” is defined herein as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, can be optionally substituted. Optionally substituted groups will be so indicated.

The following are non-limiting examples of substituents which can substitute for hydrogen atoms on a moiety: halogen (chlorine (Cl), bromine (Br), fluorine (F) and iodine(I)), —CN, —NO₂, oxo (═O), —OR¹¹, —SR¹¹, —N(R¹¹)₂, —NR¹¹C(O)R¹¹, —SO₂R¹¹, —SO₂OR¹¹, —SO₂N(R¹¹)₂, —C(O)R¹¹, —C(O)OR¹¹, —C(O)N(R¹¹)₂, C₁₋₆ alkyl, C₁₋₆ haloalkyl, C₁₋₆ alkoxy, C₂₋₈ alkenyl, C₂₋₈ alkynyl, C₃₋₁₄ cycloalkyl, aryl, heterocycle, or heteroaryl, wherein each of the alkyl, haloalkyl, alkenyl, alkynyl, alkoxy, cycloalkyl, aryl, heterocycle, and heteroaryl groups is optionally substituted with 1-10 (e.g., 1-6 or 1-4) groups selected independently from halogen, —CN, —NO₂, oxo, and R¹¹; wherein R¹¹, at each occurrence, independently is hydrogen, —OR¹², —SR¹², —C(O)R¹², —C(O)OR¹², —C(O)N(R¹²)₂, —SO₂R¹², —S(O)₂OR¹², —N(R¹²)₂, —NR¹²C(O)R¹², C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R¹¹ units taken together with the atom(s) to which they are bound form an optionally substituted carbocycle or heterocycle wherein said carbocycle or heterocycle has 3 to 7 ring atoms; wherein R¹², at each occurrence, independently is hydrogen, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R¹² units taken together with the atom(s) to which they are bound form an optionally substituted carbocycle or heterocycle wherein said carbocycle or heterocycle preferably has 3 to 7 ring atoms.

In certain embodiments, the substituents are selected from the group consisting of:
i) —OR¹³; for example, —OH, —OCH₃, —OCH₂CH₃, —OCH₂CH₂CH₃;
ii) —C(O)R¹³; for example, —COCH₃, —COCH₂CH₃, —COCH₂CH₂CH₃;
iii) —C(O)OR¹³; for example, —CO₂CH₃, —CO₂CH₂CH₃, —CO₂CH₂CH₂CH₃;
iv) —C(O)N(R¹³)₂; for example, —CONH₂, —CONHCH₃, —CON(CH₃)₂;
v) —N(R¹³)₂; for example, —NH₂, —NHCH₃, —N(CH₃)₂, —NH(CH₂CH₃);
vi) halogen: —F, —Cl, —Br, and —I;
vii) —CHₑX_{g}; wherein X is halogen, m is from 0 to 2, e+g=3; for example, —CH₂F, —CHF₂, —CF₃, —CCl₃, or —CBr₃;
viii) —SO₂R¹³; for example, —SO₂H; —SO₂CH₃; —SO₂C₆H₅;
ix) C₁-C₆ linear, branched, or cyclic alkyl;
x) Cyano
xi) Nitro;
xii) N(R¹³)C(O)R¹³;
xiii) Oxo (═O);
xiv) Heterocycle; and
xv) Heteroaryl,
   wherein each R¹³ is independently hydrogen, optionally substituted C₁-C₆ linear or branched alkyl (e.g., optionally substituted C₁-C₄ linear or branched alkyl), or optionally substituted C₃-C₆ cycloalkyl (e.g optionally substituted C₃-C₄ cycloalkyl); or two R¹³ units can be taken together to form a ring comprising 3-7 ring atoms. In certain aspects, each R¹³ is independently hydrogen, C₁-C₆ linear or branched alkyl optionally substituted with halogen or C₃-C₆ cycloalkyl or C₃-C₆ cycloalkyl.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆, alkyl.

In all of the embodiments provided herein, examples of suitable optional substituents are not intended to limit the scope of the claimed invention. The compounds of the invention may contain any of the substituents, or combinations of substituents, provided herein.

For the purposes of the invention the terms “compound,” “analog,” and “composition of matter” stand equally well for the novel functionalized 1,3 -benzenediols described herein, including all enantiomeric forms, diastereomeric forms, salts, and the like, and the terms “compound,” “analog,” and “composition of matter” are used interchangeably throughout the present specification.

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

When any variable occurs more than one time in any constituent or in any formula, its definition in each occurrence is independent of its definition at every other occurrence (e.g., in N(R¹²)₂, each R¹² may be the same or different than the other). Combinations of substituents and/or variables are permissible only if such combinations result in stable compounds.

The terms “treat” and “treating” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

### Compounds of the Invention

The invention further comprises functionalized 1,3 -benzenediols effective for treating or preventing inflammation and pain. The 1,3 -benzenediols of the invention are preferably compounds of formula (I):

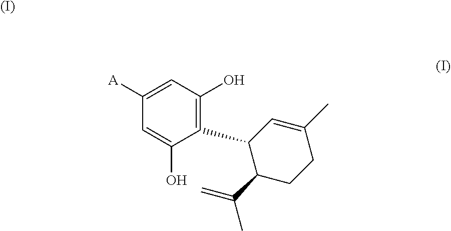

including hydrates, solvates, pharmaceutically acceptable salts, prodrugs and complexes thereof, wherein:
A is selected from the group consisting of

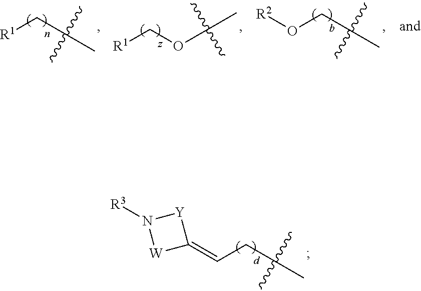

z is 0, 1, or 2;
when A is

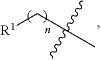

R¹ is selected from the group consisting of

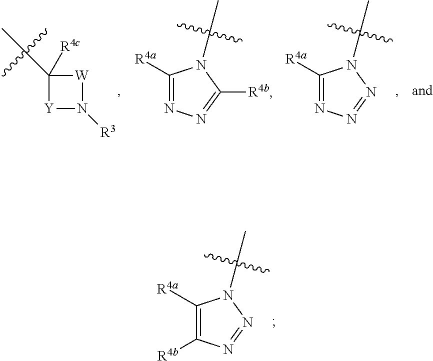

when A is

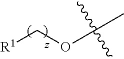

and z is 0, R¹ is

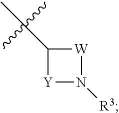

when A is

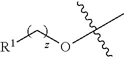

and z is 1, R¹ is

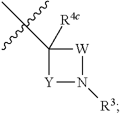

when A is

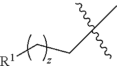

and z is 2, R¹ is selected from the group consisting of

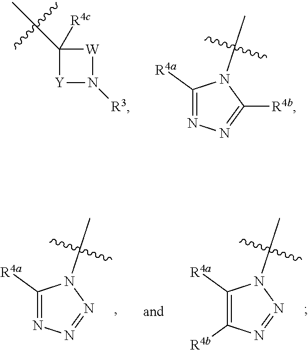

when R¹ is

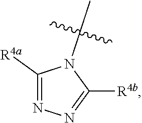

n is not 0;
when R¹ is

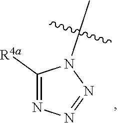

n is not 0;
when R¹ is

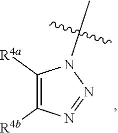

n is not 0;
R² is

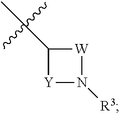

W is (CH₂)ₘ;
m is 1 or 2;
Y is (CH₂)_{q};
q is 1 or 2;
n is 0, 1, 2, or 3;
b is 0, 1, 2, or 3;
d is 0, 1, 2, or 3;
R³ is selected from the group consisting of COR⁵, CO₂R⁶, CONR^{7a}R^{7b}, SO₂NR^{7a}R^{7b}, SO₂R⁸, and optionally substituted heteroaryl;
R^{4a} and R^{4b} are each independently selected from the group consisting of hydrogen and C₁₋₆ alkyl;
R^{4c} is selected from the group consisting of hydrogen and OH;
R⁵ is selected from the group consisting of hydrogen, unsubstituted C₁₋₆ alkyl, substituted C₁₋₆ alkyl, unsubstituted heteroaryl, substituted heteroaryl, —C(R^{9a}R^{9b})NR^{7a}R^{7b}, and —C(R^{9a}R^{9b})OR¹⁰;
R⁶ is unsubstituted C₁₋₆ alkyl or substituted C₁₋₆ alkyl;
R^{7a} and R^{7b} are each independently selected from the group consisting of hydrogen, unsubstituted C₁₋₆ alkyl and substituted C₁₋₆ alkyl;
R⁸ is selected from the group consisting of hydrogen, unsubstituted C₁₋₆ alkyl, substituted C₁₋₆ alkyl, unsubstituted heteroaryl and substituted heteroaryl;
R^{9a} and R^{9b} are each independently selected from the group consisting of hydrogen, C₁₋₆ alkyl, C₃₋₇ branched alkyl, CH₂OH, CH(OH)CH₃, CH₂Ph, CH₂(4-OH-Ph), (CH₂)₄NH₂, (CH₂)₃NHC(NH₂)NH, CH₂(3-indole), CH₂(5-imidazole), CH₂CO₂H, CH₂CH₂CO₂H, CH₂CONH₂, and CH₂CH₂CONH₂; and
R¹⁰ is selected from the group consisting of hydrogen and C₁₋₆ alkyl.

The compounds of the invention further include enantiomers of compounds of the formula (I).

The compounds of the invention further include compounds of the formula (I) that are isotopically labeled with 1 to 10 deuterium atoms.

The compounds of the invention further include compounds having formula (II):

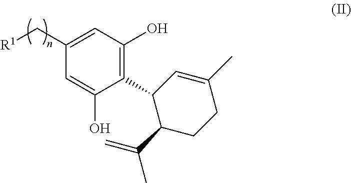

including hydrates, solvates, pharmaceutically acceptable salts, and complexes thereof, wherein R¹ and n of formula (II) are as defined above with respect to formula (I). In certain embodiments of the invention, R¹ and n of formula (II) are as defined in Table 1 below.

TABLE 1

| Entry | R¹ | n |
|---|---|---|
| 1 | 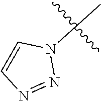 [SMILES: *n1ccnn1] | 1 |
| 2 | 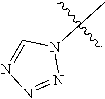 [SMILES: *n1cnnn1] | 1 |
| 3 | 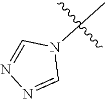 [SMILES: *n1cnnc1] | 1 |
| 4 | 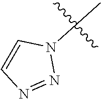 [SMILES: *n1ccnn1] | 2 |
| 5 | 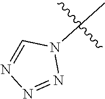 [SMILES: *n1cnnn1] | 2 |
| 6 | 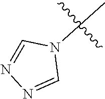 [SMILES: *n1cnnc1] | 2 |

The compounds of the invention further include enantiomers of compounds of the formula (II).

The compounds of the invention further include compounds of the formula (II) that are isotopically labeled with 1 to 10 deuterium atoms.

The compounds of the invention further include compounds having formula (III):

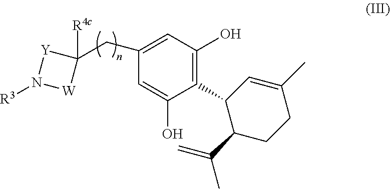

including hydrates, solvates, pharmaceutically acceptable salts, and complexes thereof, wherein R³, R^{4c}, Y, W, and n of formula (III) are as defined above with respect to formula (I). In certain embodiments of the invention, R³, R^{4c}, Y, W, and n of formula (III) are as defined in Table 2 below.

TABLE 2

| Entry | R³ | R^{4c} | Y | W | n |
|---|---|---|---|---|---|
| 1 | COCH₃ | H | CH₂ | CH₂ | 1 |
| 2 | CO₂CH₃ | H | CH₂ | CH₂ | 1 |
| 3 | CO₂CH₂CH₃ | H | CH₂ | CH₂ | 1 |
| 4 | CON(CH₃)₂ | H | CH₂ | CH₂ | 1 |
| 5 | SO₂N(CH₃)₂ | H | CH₂ | CH₂ | 1 |
| 6 | SO₂CH₃ | H | CH₂ | CH₂ | 1 |
| 7 | COCH₃ | H | CH₂ | CH₂CH₂ | 1 |
| 8 | CO₂CH₃ | H | CH₂ | CH₂CH₂ | 1 |
| 9 | CO₂CH₂CH₃ | H | CH₂ | CH₂CH₂ | 1 |
| 10 | CON(CH₃)₂ | H | CH₂ | CH₂CH₂ | 1 |
| 11 | SO₂N(CH₃)₂ | H | CH₂ | CH₂CH₂ | 1 |
| 12 | SO₂CH₃ | H | CH₂ | CH₂CH₂ | 1 |
| 13 | COCH₃ | H | CH₂CH₂ | CH₂ | 1 |
| 14 | CO₂CH₃ | H | CH₂CH₂ | CH₂ | 1 |
| 15 | CO₂CH₂CH₃ | H | CH₂CH₂ | CH₂ | 1 |
| 16 | CON(CH₃)₂ | H | CH₂CH₂ | CH₂ | 1 |
| 17 | SO₂N(CH₃)₂ | H | CH₂CH₂ | CH₂ | 1 |
| 18 | SO₂CH₃ | H | CH₂CH₂ | CH₂ | 1 |
| 19 | COCH₃ | H | CH₂CH₂ | CH₂CH₂ | 1 |
| 20 | CO₂CH₃ | H | CH₂CH₂ | CH₂CH₂ | 1 |
| 21 | CO₂CH₂CH₃ | H | CH₂CH₂ | CH₂CH₂ | 1 |
| 22 | CON(CH₃)₂ | H | CH₂CH₂ | CH₂CH₂ | 1 |
| 23 | SO₂N(CH₃)₂ | H | CH₂CH₂ | CH₂CH₂ | 1 |
| 24 | SO₂CH₃ | H | CH₂CH₂ | CH₂CH₂ | 1 |

The compounds of the invention further include enantiomers of compounds of the formula (III).

The compounds of the invention further include compounds of the formula (III) that are isotopically labeled with 1 to 10 deuterium atoms.

The compounds of the invention further include compounds having formula (IV):

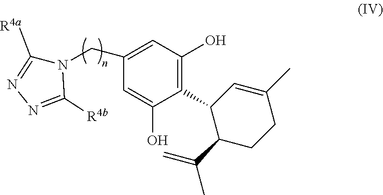

including hydrates, solvates, pharmaceutically acceptable salts, and complexes thereof, wherein n, R^{4a} and R^{4b} are as defined above with respect to formula (I).

The compounds of the invention further include enantiomers of compounds of the formula (IV).

The compounds of the invention further include compounds of the formula (IV) that are isotopically labeled with 1 to 10 deuterium atoms.

The compounds of the invention further include compounds having formula (V):

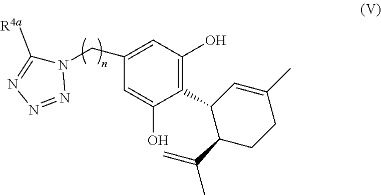

including hydrates, solvates, pharmaceutically acceptable salts, and complexes thereof, wherein n and R^{4a} are as defined above with respect to formula (I).

The compounds of the invention further include enantiomers of compounds of the formula (V).

The compounds of the invention further include compounds of the formula (V) that are isotopically labeled with 1 to 10 deuterium atoms.

The compounds of the invention further include compounds having formula (VI):

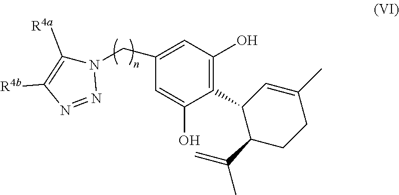

including hydrates, solvates, pharmaceutically acceptable salts, and complexes thereof, wherein n, R^{4a} and R^{4b} are as defined above with respect to formula (I).

The compounds of the invention further include compounds of the formula (VI) that are isotopically labeled with 1 to 10 deuterium atoms.

The compounds of the invention further include enantiomers of compounds of the formula (VI).

The compounds of the invention further include compounds having formula (VII):

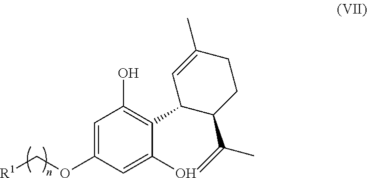

including hydrates, solvates, pharmaceutically acceptable salts, and complexes thereof, wherein R¹ and z are as defined above with respect to formula (I).

The compounds of the invention further include enantiomers of compounds of the formula (VII).

The compounds of the invention further include compounds of the formula (VII) that are isotopically labeled with 1 to 10 deuterium atoms.

The compounds of the invention further include compounds having formula (VIII):

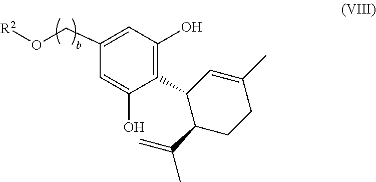

including hydrates, solvates, pharmaceutically acceptable salts, and complexes thereof, wherein R² and b are as defined above with respect to formula (I).

The compounds of the invention further include enantiomers of compounds of the formula (VIII).

The compounds of the invention further include compounds of the formula (VIII) that are isotopically labeled with 1 to 10 deuterium atoms.

The compounds of the invention further include compounds having formula (IX):

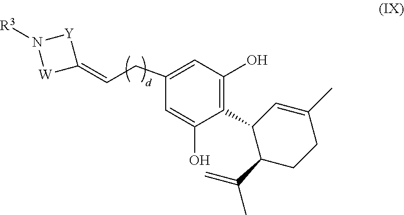

including hydrates, solvates, pharmaceutically acceptable salts, and complexes thereof, wherein R³, Y, W, and d of formula (IX) are as defined above with respect to formula (I). In certain embodiments of the invention, R³, Y, W, and d of formula (IX) are as defined in Table 3 below.

TABLE 3

| Entry | R³ | Y | W | d |
|---|---|---|---|---|
| 1 | COCH₃ | CH₂ | CH₂ | 1 |
| 2 | CO₂CH₃ | CH₂ | CH₂ | 1 |
| 3 | CO₂CH₂CH₃ | CH₂ | CH₂ | 1 |
| 4 | CON(CH₃)₂ | CH₂ | CH₂ | 1 |
| 5 | SO₂N(CH₃)₂ | CH₂ | CH₂ | 1 |
| 6 | SO₂CH₃ | CH₂ | CH₂ | 1 |
| 7 | COCH₃ | CH₂ | CH₂CH₂ | 1 |
| 8 | CO₂CH₃ | CH₂ | CH₂CH₂ | 1 |
| 9 | CO₂CH₂CH₃ | CH₂ | CH₂CH₂ | 1 |
| 10 | CON(CH₃)₂ | CH₂ | CH₂CH₂ | 1 |
| 11 | SO₂N(CH₃)₂ | CH₂ | CH₂CH₂ | 1 |
| 12 | SO₂CH₃ | CH₂ | CH₂CH₂ | 1 |
| 13 | COCH₃ | CH₂CH₂ | CH₂ | 1 |
| 14 | CO₂CH₃ | CH₂CH₂ | CH₂ | 1 |
| 15 | CO₂CH₂CH₃ | CH₂CH₂ | CH₂ | 1 |
| 16 | CON(CH₃)₂ | CH₂CH₂ | CH₂ | 1 |
| 17 | SO₂N(CH₃)₂ | CH₂CH₂ | CH₂ | 1 |
| 18 | SO₂CH₃ | CH₂CH₂ | CH₂ | 1 |
| 19 | COCH₃ | CH₂CH₂ | CH₂CH₂ | 1 |
| 20 | CO₂CH₃ | CH₂CH₂ | CH₂CH₂ | 1 |
| 21 | CO₂CH₂CH₃ | CH₂CH₂ | CH₂CH₂ | 1 |
| 22 | CON(CH₃)₂ | CH₂CH₂ | CH₂CH₂ | 1 |
| 23 | SO₂N(CH₃)₂ | CH₂CH₂ | CH₂CH₂ | 1 |
| 24 | SO₂CH₃ | CH₂CH₂ | CH₂CH₂ | 1 |

The compounds of the invention further include enantiomers of compounds of the formula (IX).

The compounds of the invention further include compounds of the formula (IX) that are isotopically labeled with 1 to 10 deuterium atoms.

The compounds of the invention further include compounds having formula (X):

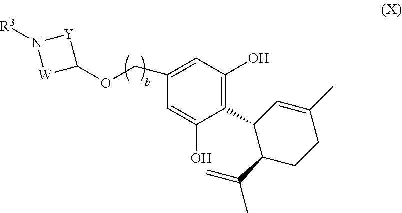

including hydrates, solvates, pharmaceutically acceptable salts, and complexes thereof, wherein R³, Y, W, and b of formula (X) are as defined above with respect to formula (I). In certain embodiments of the invention, R³, Y, W, and b of formula (X) are as defined in Table 4 below.

TABLE 4

| Entry | R³ | Y | W | b |
|---|---|---|---|---|
| 1 | COCH₃ | CH₂ | CH₂ | 1 |
| 2 | CO₂CH₃ | CH₂ | CH₂ | 1 |
| 3 | CO₂CH₂CH₃ | CH₂ | CH₂ | 1 |
| 4 | CON(CH₃)₂ | CH₂ | CH₂ | 1 |
| 5 | SO₂N(CH₃)₂ | CH₂ | CH₂ | 1 |
| 6 | SO₂CH₃ | CH₂ | CH₂ | 1 |
| 7 | COCH₃ | CH₂ | CH₂CH₂ | 1 |
| 8 | CO₂CH₃ | CH₂ | CH₂CH₂ | 1 |
| 9 | CO₂CH₂CH₃ | CH₂ | CH₂CH₂ | 1 |
| 10 | CON(CH₃)₂ | CH₂ | CH₂CH₂ | 1 |
| 11 | SO₂N(CH₃)₂ | CH₂ | CH₂CH₂ | 1 |
| 12 | SO₂CH₃ | CH₂ | CH₂CH₂ | 1 |
| 13 | COCH₃ | CH₂CH₂ | CH₂ | 1 |
| 14 | CO₂CH₃ | CH₂CH₂ | CH₂ | 1 |
| 15 | CO₂CH₂CH₃ | CH₂CH₂ | CH₂ | 1 |
| 16 | CON(CH₃)₂ | CH₂CH₂ | CH₂ | 1 |
| 17 | SO₂N(CH₃)₂ | CH₂CH₂ | CH₂ | 1 |
| 18 | SO₂CH₃ | CH₂CH₂ | CH₂ | 1 |
| 19 | COCH₃ | CH₂CH₂ | CH₂CH₂ | 1 |
| 20 | CO₂CH₃ | CH₂CH₂ | CH₂CH₂ | 1 |
| 21 | CO₂CH₂CH₃ | CH₂CH₂ | CH₂CH₂ | 1 |
| 22 | CON(CH₃)₂ | CH₂CH₂ | CH₂CH₂ | 1 |
| 23 | SO₂N(CH₃)₂ | CH₂CH₂ | CH₂CH₂ | 1 |
| 24 | SO₂CH₃ | CH₂CH₂ | CH₂CH₂ | 1 |

The compounds of the invention further include enantiomers of compounds of the formula (X).

The compounds of the invention further include compounds of the formula (X) that are isotopically labeled with 1 to 10 deuterium atoms.

For the purposes of demonstrating the manner in which the compounds of the invention are named and referred to herein, the compound having the formula:

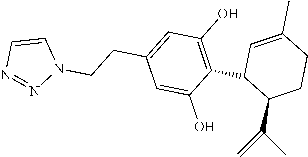

has the chemical name 5-(2-(1H-1,2,3-triazol-1-yl)ethyl)-2-((1R,6R)-3-methyl-6-(prop-1-en-2-yl)cyclohex-2-enyl)benzene-1,3-diol, and is sometimes referred to herein as KLS-13007.

For the purposes of demonstrating the manner in which the compounds of the invention are named and referred to herein, the compound having the formula:

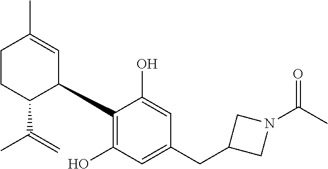 [SMILES: C=C(C)[C@@H]1CCC(C)=C[C@H]1c1c(O)cc(CC2CN(C(C)=O)C2)cc1O]

has the chemical name 1-(3-(3,5-dihydroxy-4-((1R,6R)-3-methyl-6-(prop-1-en-2-yl)cyclohex-2-enyl)benzyl)azetidin-1-yl)ethenone, and is sometimes referred to herein as KLS-13019.

For the purposes of demonstrating the manner in which the compounds of the invention are named and referred to herein, the compound having the formula:

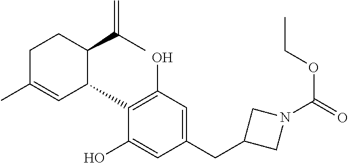 [SMILES: C=C(C)[C@@H]1CCC(C)=C[C@H]1c1c(O)cc(CC2CN(C(=O)OCC)C2)cc1O]

has the chemical name ethyl 3-(3,5-dihydroxy-4-((1R,6R)-3-methyl-6-(prop-1-en-2-yl)cyclohex-2-enyl)benzyl)azetidine-1-carboxylate, and is sometimes referred to herein as KLS-13022.

### Compositions of the Invention

The present invention also relates to compositions or formulations which comprise the functionalized 1,3-benzenediols according to the invention. In general, the compositions of the invention comprise an effective amount of at least one functionalized 1,3-benzenediol and/or a salt thereof and at least one excipient.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the invention have improved cellular potency, pharmacokinetic properties, as well as improved oral bioavailability.

Examples of suitable excipients are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, PA (1985), the entire disclosure of which is incorporated by reference herein for all purposes.

As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the invention can be administered topically or parenterally, neat or in combination with conventional pharmaceutical carriers. Applicable solid carriers can include one or more substances which can also act as lubricants, solubilizers, suspending agents, fillers, glidants, compression aids, binders or disintegrating agents, or encapsulating materials. The compounds can be formulated in conventional manner, for example, in a manner similar to that used for known therapeutic agents. In powders, the carrier can be a finely divided solid, which is an admixture with a finely divided compound.

Liquid carriers can be used in preparing solutions, suspensions and emulsions. A compound of the invention can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, suspending agents, thickening agents, colors, viscosity regulators, stabilizers, and osmo-regulators. Examples of liquid carriers for topical and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection.

In certain embodiments, the pharmaceutical composition is in unit dosage form. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Such unit dosage form can contain from about 1 mg/kg of compound to about 500 mg/kg of compound, and can be given in a single dose or in two or more doses. Such doses can be administered in any manner useful in directing the compound(s) to the recipient's skin or ocular tissue, including topically or parenterally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the invention can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

Compounds described herein can be administered parenterally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions and solutions.

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable.

To increase the effectiveness of compounds of the invention, it can be desirable to combine a compound with other agents effective in the treatment of the target disorder. For example, other active compounds (i.e., other active ingredients or agents) effective in treating the target disorder can be administered with compounds of the invention. The other agents can be administered at the same time or at different times than the compounds disclosed herein.

Compounds and compositions of the invention can be useful for the treatment or inhibition of a pathological condition or disorder in a mammal, for example, a human subject.

Non-limiting examples of compositions according to the invention include from about 0.001 mg to about 1000 mg of one or more functionalized 1,3-benzene diols according to the present invention and one or more excipients; from about 0.01 mg to about 100 mg of one or more functionalized 1,3-benzenediols according to the invention and one or more excipients; and from about 0.1 mg to about 10 mg of one or more functionalized 1,3-benzenediols according to the invention; and one or more excipients.

### Method of the Invention

The method of the invention comprises the administration of compounds and/or compositions of the invention to prevent or treat inflammation and/or pain. The activity of the compounds and compositions is mediated in part through antagonism of GPR55 on DRG neurons.

Accordingly, the method of the invention is useful in the treatment of any inflammatory disease that has GPR55 expressed in the target organ(s) of the disease. Conditions that can be treated by the method of the invention include but are not limited to chemotherapy-induced peripheral neuropathy (CIPN), traumatic brain injury, a traumatic spinal cord injury, stroke, autoimmune diseases, viral infections, surgical trauma, osteoarthritis and chronic opioid treatment. The specific scope of treatment is defined by the presence of the GPR55 receptors in target cells/tissues that mediate the inflammatory responses.

The concept that a single molecular entity exhibits high potency neuroprotective and anti-inflammatory actions is not obvious from the literature in that there is no specific drug or regimen currently available for the treatment of any neurodegenerative or neuropathic disease with an observed neuroinflammatory component. Without wishing to be bound by any theory, the invention is believed to produce neuroprotection through the regulation of calcium homeostasis imparted through the action on a mitochondrial Na⁺Ca⁺ exchanger (Brenneman et al., 2019) and anti-inflammatory actions through the action of GPR55 receptor antagonism.

The invention will be illustrated in more detail with reference to the following Examples, but it should be understood that the invention is not deemed to be limited thereto.

### EXAMPLES

Functionalized 1,3-benzene diols will be shown to exhibit anti-inflammatory responses in cultures derived from dorsal root ganglion, an entity that exhibits vulnerability to neuronal damage from chemotherapeutic agents and long-term inflammatory responses that result in pain and neural damage (Han and Smith, 2013). Therapeutic activity will be demonstrated in an animal model of chemotherapy-induced peripheral neuropathy in a dose-dependent and orally effective regimen with an exemplary functionalized 1,3-benzene diol (KLS-13019).

In previous studies, KLS-13019 was shown to prevent oxidative stress and nerve cell damage in cultures of the central and peripheral nervous system (Kinney et al., 2016; Brenneman et al., 2018; Brenneman et al., 2019; U.S. Pat. Nos. 9,611,213 B2; and 10,004,722 B2). These protective effects from free radical and excessive calcium overload mediated by ethanol or paclitaxel could be prevented by an inhibitor or the sodium-calcium exchanger-1. In confirmative studies, a decrease in the gene expression of the mitochondrial sodium-calcium exchanger-1 with siRNA resulted in an inhibition of the protective effect of KLS-13019. These studies confirmed the protective mechanism that is established for hippocampal and dorsal root ganglion neurons that may be pertinent to the regulation of oxidative stress in other tissues.

In this disclosure, the reversal of an established increase in inflammatory markers will be demonstrated in cultures of dorsal root ganglion, a tissue that is known to be associated with neuropathic pain and CIPN (Krames, 2014).

Relevant to the present disclosure, increases in IL-1β and IL-6 are shown to be prevented in dissociated DRG neurons after treatment with KLS -13019. Both IL-β and IL-6 have been identified to be among the identifiable mediators of inflammation in patients exhibiting neuropathic pain (Matsuda et al., 2019).

GPR55, a putative endocannabinoid receptor, has been associated with inflammatory and neuropathic pain (Gangadharan et al., 2013; Guerrero-Alba et al., 2019). Furthermore, GPR55 knockout mice have been shown to be resistant to induced inflammatory allodynia produced by Freund's complete adjuvant and partial nerve ligation (Staton et al 2008). Evidence in this disclosure demonstrates that GPR55 is increased after paclitaxel treatment which contributes to increases in inflammatory mediators (IL-1β and IL-6) in sensory neurons. Further evidence has demonstrated that treatment with LPIA (lysophosphatidylinositol arachidonate), the endogenous agonist for GPR55, can produce increases in neuronal IL-1β and IL-6 in dorsal root ganglion cultures. KLS -13019 is now shown to block LPIA-induced increases in GPR55 immunoreactive area in DRG cultures. Furthermore, KLS-13019 has an antagonistic action on LPIA stimulated β-arrestin in a model system of DiscoverX cells expressing human GPR55. These studies support a pro-nociceptive, pro-inflammatory role for GPR55 that mediate both an acute and chronic effects in DRG. The GPR55 target is believed to be complementary to our previous studies with the NCX-1 target which exhibited acute regulation of mitochondrial calcium levels by extrusion of excess calcium in DRG (Brenneman et al, 2019). Our data suggest a bi-modal pharmacological effect of KLS-13019 that can both increase viability of sensory neurons exposed to paclitaxel and antagonize GPR55 that can mediate long-term neuroinflammatory and sensory neuron damage that may contribute to neuropathic pain.

An emerging concept is that chemotherapeutic agents (including paclitaxel) promote inflammatory responses through activation of the NLRP3 inflammasome (Zeng et al., 2019). Although paclitaxel is believed to be among the substances that can drive “priming” for signal-mediated events needed for the activation of inflammasome-mediated assembly in macrophages, our new findings indicate that this action of paclitaxel is more complex in DRG neurons. Importantly, a novel finding of the present disclosure is that paclitaxel treatment can also increase the expression of GPR55 in DRG cultures. Thus, an alternative explanation for paclitaxel-mediated “priming” is herein described: the concept is that GPR55 is the mediator of paclitaxel-induced “priming” of NLRP3 inflammasome assembly. With this finding, GPR55 is a “priming” signal for inflammasome assembly, in addition to paclitaxel. Our new data strongly indicate that GPR55 is a sufficient priming signal in DRG cultures to mediate all of the increases in IL-1β, which is known to be elevated through the action of the NLRP3 inflammasome. Because paclitaxel can also produce toxic increases in mitochondrial calcium and elevated reactive oxygen species in sensory neurons, we have concluded that these actions of paclitaxel on the activation of the inflammasome complex are the major actions produced by paclitaxel in sensory neurons. Thus, two roles of paclitaxel are revealed in DRG neurons: 1) increases in the expression of GPR55; and 2) increases ROS and calcium in the mitochondria. The rationale for KLS-13019 mediating anti-inflammatory actions produced by paclitaxel is that this agent is a GPR55 antagonist that can block the GPR55-mediated “priming” of NLRP3 inflammasome assembly. The KLS-13019-mediated antagonism of GPR55 actions is supported through effects observed on decreasing levels of IL-1β, IL-6 and NLRP3 in DRG neurons. Evidence follows that KLS-13019 is completely effective in reducing paclitaxel- and LPIA-mediated increases in these inflammatory markers back to that of control levels.

### Procedures

The following procedures can be utilized in evaluating and selecting compounds that are both protective from oxidative stress and anti-inflammatory as an antagonist to GPR55 for the treatment of pain.

### A. Cell Cultures

Primary neuronal cultures were utilized to establish the protective and anti- inflammatory properties of 1,3-benzene diols in neuronal models that are relevant to treatments for neuropathic pain. Because of the recognized importance of sensory neurons in DRG to transmitting peripheral nociception (Krames, 2014) and the reported accumulation of paclitaxel in DRG from models of CIPN (Duggett et al., 2016), the preparation of choice for the in vitro studies to study protection and anti-inflammation was dissociated cultures from rat DRG. DRG cultures derived from embryonic day 18 rats were employed as the primary assay system to study KLS-13019-properties. In brief, DRG were obtained commercially through Brain Bits (Springfield, IL) and cultures prepared with slight modifications to methods previously described (Brenneman et al., 2019). Tissue was dissociated with a papain-based kit from Worthington Biochemical Corporation (Lakewood, NJ). The DRG cells were plated at low density (10,000 cells/well) in a 96-well format and maintained in serum-free medium consisting of Neurobasal Medium supplemented with B27, GlutaMAX (Gibco) and 25 ng/ml Nerve Growth Factor. Poly-D-lysine coated plates (BD Biosciences, Franklin Lakes, NJ) were employed for this culture system. Prior to the initiation of experiments between days 5 and 9 in vitro, a complete change of medium was performed in a working volume of 100 μL.

Because the scope of the anti-inflammatory actions of the 1,3-benzene diols included the central nervous system as well, hippocampal cultures were also employed as they express both mNCX-1 and GPR55, the two targets that mediate protection and inflammation, respectively. Dissociated hippocampal cultures derived from embryonic day 18 rats were employed as the primary screening system to test for inflammatory action of KLS-13019 for the CNS. In brief, hippocampal tissue was obtained commercially through Brain Bits (Springfield, IL) and cultures prepared with slight modifications to methods previously described (Brenneman et al, 2018). Tissue was dissociated with a papain-based kit from Worthington Biochemical Corporation (Lakewood, NJ). The hippocampal cells were plated at low density (10,000 cell/well) in a 96-well format and maintained in serum-free medium consisting of Neurobasal Medium supplemented with B27 and GlutaMAX (Life Technologies, Carlsbad, CA). Poly-L-lysine-coated plates (BD Biosciences, Franklin Lakes, NJ) were used because of the preferential adherence and survival of neurons on this matrix support. Prior to the initiation of all experiments between days 12 and 19 in vitro, a complete change of medium was performed in a working volume of 100 μL.

### B. Immunocytochemistry

To assess all antibody-based assays, immunocytochemical methods were employed. The goals for these assays included: 1) to assess the immunoreactive area of our molecular targets (IL-1β, IL-6, GPR55, NLRPA3) with their respective primary antibodies and distinctively labeled secondary antibodies; and 2) to compare the relative responses in both neuronal cell bodies and neurites. Prior to fixation, growth medium was removed and the wells were rinsed one time with 100 μL of DPBS (37° C.). This warm rinse is particularly important to maintain structural stability of neurites. After removal of the DPBS, cultures were fixed for 20 min at room temperature with 50 μL/well of 3.5% formaldehyde (Sigma-Aldrich: 252549) in warm (37° C.) DPBS that contained 5.5 μg/mL of Hoechst 33342 dye (Invitrogen: H3570) to label cell nuclei. After removal of the fixative, the cultures were rinsed twice with 100 μL of DPBS and then a permeabilization/blocking buffer containing 5% normal goat serum and 0.3% triton-X100 in DPBS was added to the cultures for 10 min. After removal of the blocking buffer, the cultures were rinsed twice with 100 μL of DPBS and then primary antibodies were added for one-hour incubation at room temperature. Neurons were identified with antiserum to type III beta tubulin (tuj 1) to measure changes in all neuronal parameters. The primary antiserum employed was rabbit anti-rat obtained from Sigma-Aldrich (T2200) and used at 1:200 dilution. The secondary was an Alexa Fluor 488-conjugated Fab fragment of goat anti-rabbit IgG obtained from Life Technologies (A11070) used at 1:600. After the secondary antibody treatment, cultures were rinsed 3 times with 100 μL of DPBS before performing high content fluorescent analysis. For storage, the wells were placed in 100 μL of sterile DPBS, with the plates wrapped in aluminum foil and maintained at 4° C. The same conditions were employed for DRG and hippocampal cultures. For the detection of inflammatory markers, the following primary antibodies were used: for IL-1β (PA5-88078); for IL-6 (PA1-26811); for NLRP3 (PA5-7940); and for GPR55 (ab203663). All primary antibodies for cytokines were obtained from Life Technologies. The GPR55 antibody was obtained from abcam. All primary antibodies were diluted 1:250 and all secondary antibodies were used at 1:600. The secondary antibodies were obtained from Life Technologies. The following dyes labeled the secondary antibodies: Alexa Fluor 488, 555, 687 and 750 nm. By using secondary antibodies with differing dyes, the same cultures could be assayed for multiple molecular targets.

### C. High Content Image Analysis

Following the immunocytochemical assays, measurements of immunoreactive area were conducted with the Cell Insight CX5 high content imaging system (Thermo Fisher Scientific). This system is based on an inverted microscope that automatically focuses and scans fields of individual culture wells using a motorized stage at predetermined field locations. Fluorescent images from individual fields (895 μm×895 μm) were obtained with a 10×(0.30NA) Olympus objective and Photometrics X1 CCD camera, with analysis by HCS Studio 2.0 Software. The light source was LED with solid state five-color light engine used with filter sets that had the following excitation/emission: 386/440, 485/521, 560/607, 650/694 and 740/809). With this capability, multiple fluorescent assays in a single well were conducted. Images were acquired in a low-resolution mode (4×4 binning). Image analyses for neuronal cell bodies and neurites were performed with the Cellomics Neuronal Profiling BioApplication. For analysis of neurons, objects were identified as cells if they had valid nuclei and cell body measures based on size, shape and average intensity. Acceptable ranges were determined in preliminary studies to ensure that aggregated cells and non-cellular objects were excluded from the analysis.

The primary goal was to examine the immunoreactive spot area for various cellular targets on the neurons of the cultures, with the objective being the comparison of cell bodies and neurites. Imaging parameters were empirically determined for both DRG and hippocampal cultures. Type III beta tubulin immunoreactivity was used to identify the neurons. Ten predetermined fields of view were sampled in each of 6-8 replicate wells per plate. For measuring parameters of type III beta tubulin immunoreactivity and spot analysis, the Cellomics Neuronal Profiling Bioapplication was used that combined spot analysis on neurons that resided within this Bioapplication. With this algorithm, the immunoreactive area was a relative measure that was characterized by an effective computerized spot analysis in a rapid screening mode. For each culture type, the same imaging parameters for neurons from all treatment groups were employed for their respective studies.

### D. Viability Assays

At the conclusion of each experiment, sister cultures to those assessed by image analysis were evaluated with either a fluorescent dye-based assays for neuronal viability (6-carboxyfluorescein diacetate, CFDA) or with viability dye Alamar Blue. The preferred dye was Alamar Blue as this assay of viability could be conducted and then the dye washed off the cultures for subsequent fixation and follow-up immunocytochemical assays. These two standard assays were chosen because CFDA has a specificity to measure neuronal viability and the Alamar Blue assay possessed increased sensitivity in comparison to CFDA. In addition, a portion of the reductive conversion of resazurin to the highly fluorescent resorufin in the Alamar Blue reagent is attributable to the action of mitochondrial reductases. Because of the need for enhanced sensitivity, the Alamar Blue assay was predominantly used for these studies. Similar results in assessing pharmacological responses were obtained with both assays (see Brenneman et al, 2019).

On every plate, wells without cells were used to provide a blank reading that was used to subtract background fluorescence. For the Alamar Blue viability assay, 10 ul of the dye was added directly to the culture well that contained 100 ul of nutrient medium (Ivanov et al., 2016). Incubation times with the dye ranged from 1-5 hrs., depending on the experimental goals. Fluorescence was measured at an excitation of 530 nm and an emission of 590 on a Cytofluor plate reader.

### E. β-Arrestin Assay

A commercial assay for β-arrestin was obtained from Eurofins that provided a means of testing human GPR55 in a cell line (93-024C2) that had a background of CHO-K1. In this assay, agonist-induced activation of GPR55 stimulates binding of β-arrestin to the Pro-link-tagged GPCR and forces complementation of two enzyme fragments, resulting in the formation of an active β-galactosidase enzyme. This interaction leads to an increase in enzyme activity that can be measured using chemiluminescent PathHunter detection reagents. For our application, the GPR55 agonist lysophosphatidylinositol (LPI) was tested from 0.1 nM to 30 μM to increase the relative luminescent signal relative to that of control. LPI produced a robust and reproducible signal at 10 μM. For the GPR55 antagonism assay, KLS-13019 concentrations ranging from 0.1 nM to 30 μM were tested in the presence of 16 μM LPI. Concentrations of KLS-13019 ranging from 0.1 nM to 30 μM were tested alone for possible effects of agonistic activity on β-arrestin. The data indicated that KLS-13019 had no detectible agonist activity in the β-arrestin assay.

Referring to FIG. 1, cellular viability, as measured with the viability dye Alamar Blue, is shown with relative fluorescence units (RFU) in dorsal root ganglion (DRG) cultures after treatment with 3 μM paclitaxel for 8 hours followed by 16 hours treatment with various concentrations of KLS-13019. Reference lines are provided to depict the RFU levels for control cultures (medium dash) and cultures pre-treated only with 3 μM paclitaxel (dash-dot-dash) for eight hours. Based on these data, KLS-13019 was shown to fully protect cells in dorsal root ganglion from toxicity produced by 3 μM paclitaxel back to fluorescent levels observed with control cultures. Because the KLS-13019 was not added to the cultures until after 8 hours of paclitaxel pre-treatment, these data indicate that KLS-13019-mediated protection provided by 16 hours of treatment produced a reversal of the toxic effects that had been elicited by the earlier pre-treatment with paclitaxel. To confirm, all treated cultures were continuously exposed to paclitaxel throughout the 24-hour duration of the experiment. The potency of KLS-13019 in reversing previously established paclitaxel toxicity had an EC50 of 0.2 nM±0.04 nM as determined with a four-parameter logistic analysis±the standard error. Each value is the mean of 6 determinations.

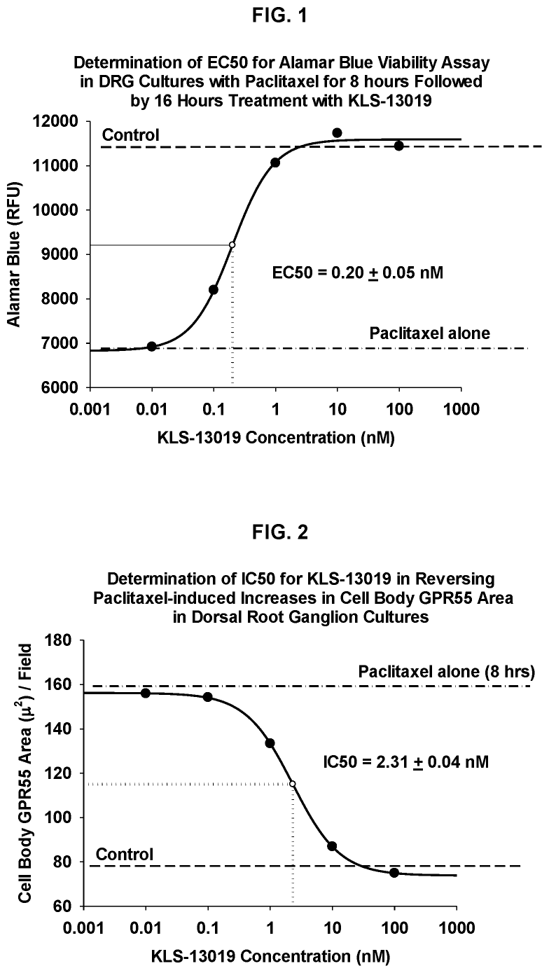

Responses of cell body GPR55 immunoreactive areas (μ²) of neurons from dorsal root ganglion (DRG) cultures are shown in FIG. 2 after pretreatment with 3 μM paclitaxel for 8 hours followed by 16 hours treatment with various concentrations of KLS-13019. Reference lines are provided to depict the average cell body GPR55 area for control cultures (medium dash) and that of cultures pre-treated only with 3 μM paclitaxel (dash-dot-dash) for eight hours. Based on these data, increases in GPR55 area in cell bodies produced by 3 μM paclitaxel were fully returned to the average area of control cultures following 16 hours treatment with KLS-13019 that were continuously co-treated with paclitaxel throughout the 24-hour duration of the experiment. Because KLS-13019 was not added to the cultures until after 8 hours of paclitaxel pre-treatment, these data indicate that 16 hours of KLS-13019 treatment resulted in a reversal of the increases in GPR55 area that had been elicited by the earlier pre-treatment with paclitaxel. The potency of KLS-13019 in reversing previously established increases in GPR55 area had an EC50 of 2.31 nM±0.24 nM as determined with a four-parameter logistic analysis±the standard error. Each value is the mean of 8 determinations. These data are consistent with the concept that increases in the proinflammatory receptor GPR55 were reversed by the high potency, high efficacy treatment with KLS -13019. These data are consistent with the conclusion that the 8 hour increases in GPR55 are reversible with KLS -13019. All assays were conducted by high content fluorescence imaging (Brenneman et al., 2018 and 2019).

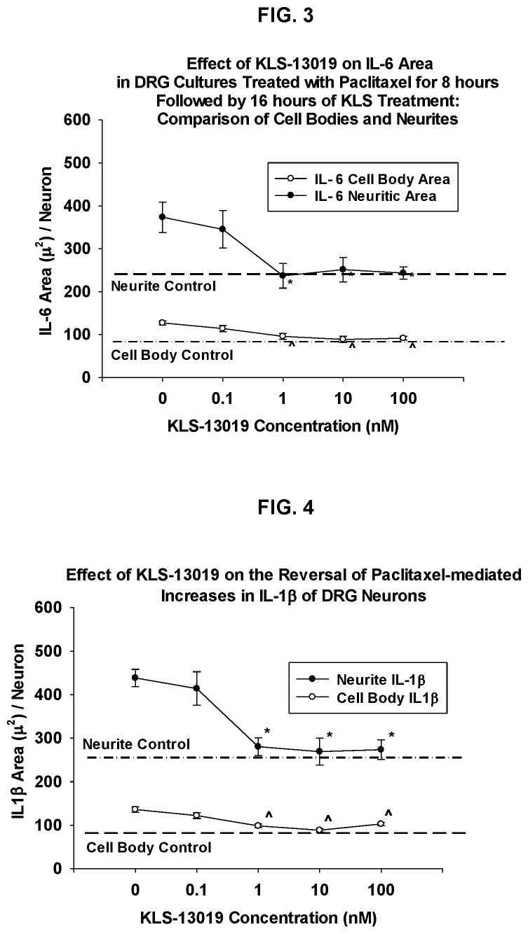

Referring to FIG. 3, responses of interleukin-6 (IL-6) immunoreactive (IR) areas (μ²) in cell bodies (open circles) and neurites (closed circles) of rat DRG neurons from dissociated cultures are shown after pretreatment with 3 μM paclitaxel for 8 hours followed by 16 hours treatment with various concentrations of KLS -13019. Reference lines are provided to depict the average cell body IL-6 area (medium dash) and neurites IL-6 (dash-dot-dash) from control cultures. The zero-concentration data point on both curves depicts values for cultures treated only with 3 μM paclitaxel. Based on these data, increases in IL-6 (IR) area in cell bodies and neurites produced by 3 μM paclitaxel were fully returned to the average area of control cultures following 16 hours treatment with KLS-13019. To confirm, all treated cultures were continuously exposed to paclitaxel throughout the 24-hour duration of the experiment. Because KLS-13019 was not added to the cultures until after 8 hours of paclitaxel pre-treatment, these data indicate that 16 hours of KLS-13019 treatment resulted in a reversal of the increases in IL-6 IR area that had been elicited by the earlier pre-treatment with paclitaxel. The potency of KLS-13019 in reversing previously established increases in IL-6 IR area had an IC50 of 0.3 nM. Each value is the mean of 8 determinations. These data are consistent with the concept that increases in the inflammatory mediator IL-6 were reversed by the high potency, high efficacy treatment with KLS-13019 for both cell bodies and neurites of the DRG neurons. These data are consistent with the conclusion that the 8 hour increases in IL-6 are reversible with KLS-13019. All assays were conducted by high content fluorescence imaging (Brenneman et al., 2018 and 2019).

Responses of interleukin-1β (IL-1β) immunoreactive (IR) areas (μ²) in cell bodies (open circles) and neurites (closed circles) of rat DRG neurons from dissociated cultures are shown in FIG. 4 after pre-treatment with 3 μM paclitaxel for 8 hours followed by 16 hours treatment with various concentrations of KLS-13019. Reference lines are provided to depict the average cell body IL-1β area (medium dash) and neurites IL-1β (dash-dot-dash) from control cultures. The zero-concentration data point on both curves depicts values for cultures treated only with 3 μM paclitaxel. Based on these data, increases in IL-1β (IR) area in cell bodies and neurites produced by 3 μM paclitaxel were fully restored to the average area of control cultures following 16 hours treatment with KLS-13019. To confirm, all treated cultures were continuously exposed to paclitaxel throughout the 24-hour duration of the experiment. Because KLS -13019 was not added to the cultures until after 8 hours of paclitaxel pre-treatment, these data indicate that 16 hours of KLS-13019 treatment resulted in a reversal of the increases in IL-1β IR area that had been elicited by the earlier pre-treatment with paclitaxel. The potency of KLS-13019 in reversing previously established increases in IL-1β IR area had an IC50 of 0.3 nM. Each value is the mean of 8 determinations. These data are consistent with the concept that increases in the inflammatory mediator IL-1β were reversed by the high potency, high efficacy treatment with KLS-13019 for both cell bodies and neurites of the DRG neurons. These data are consistent with the conclusion that the 8 hour increases in IL-1β are reversible back to control levels after treatment with KLS -13019. All assays were conducted by high content fluorescence imaging (Brenneman et al., 2018 and 2019).

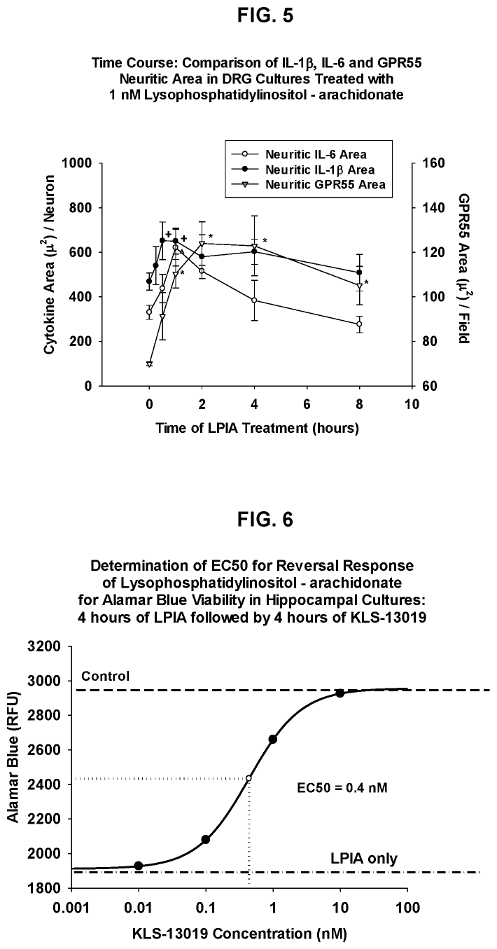

The time course of lysophosphatidylinositol-arachidonate (LPIA)-mediated effects on neuritic interleukin-1β (closed circles), interleukin-6 (open circles) and GPR55 (open inverted triangles) immunoreactive areas in DRG neurons are shown in FIG. 5. As LPIA is an identified endogenous agonist of GPR55, the effect on various proinflammatory cytokines were measured in sensory neurons of DRG cultures over the course of 8 hours with this activator of GPR55. For both neuritic IL-1β and IL-6, significant increases in IR area were observed after one hour of incubation with 1 nM LPIA. In addition, LPIA-mediated increases in GPR55 IR area were also observed after one hour of incubation in the DRG neurons. Preliminary concentration-effect studies indicated that 1 nM LPIA produced a maximum effect on GPR55 IR area in the DRG neurons. Together, these data support the conclusion that LPIA produced increases in proinflammatory cytokines as well as increases in its own receptor GPR55, thereby supporting a role for the LPIA/GPR55 system in mediating inflammation in sensory neurons.

Cellular viability, as measured with the viability dye Alamar Blue, is shown in FIG. 6 with relative fluorescence units (RFU) in hippocampal cultures after treatment with 1 nM lysophosphatidylinositol—arachidonate (LPIA for 4 hours followed by 4 hours treatment with various concentrations of KLS-13019. Reference lines are provided to depict the RFU levels for control cultures (medium dash) and cultures pre-treated only with 1 nM LPIA (dash-dot-dash) for four hours. Based on these data, KLS-13019 was shown to fully protect cells in hippocampal cultures from toxicity produced by 1 nM LPIA back to fluorescent levels observed with control cultures. Because the KLS-13019 was not added to the cultures until after 4 hours of LPIA pre-treatment, these data indicate that KLS-13019-mediated protection provided by 4 hours of treatment produced a reversal of the toxic effects that had been elicited by the earlier pre-treatment with LPIA. The potency of KLS-13019 in reversing previously established LPIA toxicity had an EC50 0.4 nM. Each value is the mean of 6 determinations. These data are consistent with the conclusion that activation of the GPR55 receptor with LPIA produced a toxicity that could be reversed after treatment with KLS-13019.

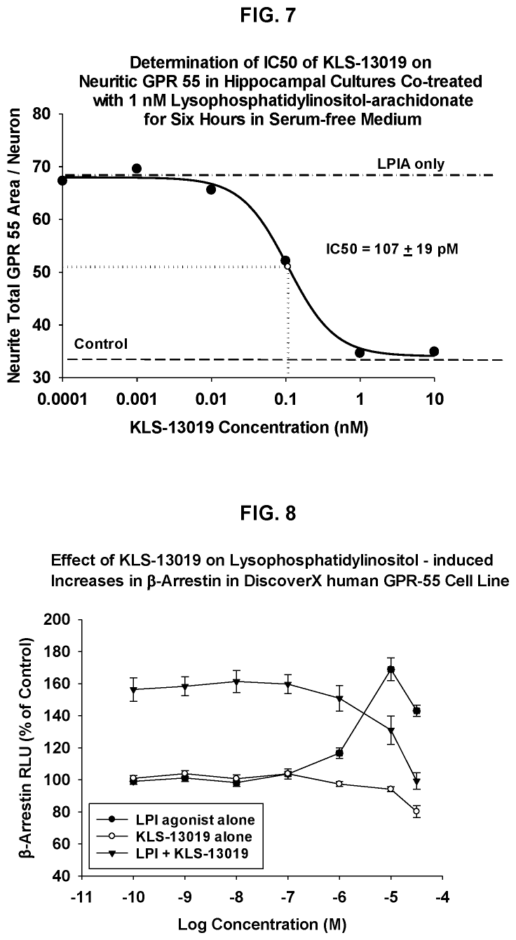

Responses of neuritic GPR55 immunoreactive areas (μ²) of neurons from dorsal root ganglion (DRG) cultures are shown in FIG. 7 after co-treatment with 1 nM LPIA and various concentrations of KLS-13019 for 6 hours. Reference lines are provided to depict the average neuritic GPR55 area for control cultures (medium dash) and that of cultures treated only with 1 nM LPIA (dash-dot-dash). Based on these data, the GPR55 agonist, LPIA, produced an increase in neuritic GPR55 that could be blocked by co-treatment with KLS-13019 over the course of 6 hours. The potency of KLS-13019 in preventing increases in GPR55 area had an IC50 of 107±19 pM as determined with a four-parameter logistic analysis±the standard error. Each value is the mean of 8 determinations. These data are consistent with the conclusion that LPIA-mediated increases in the proinflammatory receptor GPR55 can be prevented by the high potency, high efficacy treatment with KLS-13019. All assays were conducted by high content fluorescence imaging (Brenneman et al., 2018 and 2019).

A PathHunter β-arrestin assay is shown in FIG. 8 that provides a measure of an antagonist mode assessment of KLS-13019 with a human GPR55 model system (Eurofins). To measure the β-arrestin response to a GPR55 agonist, various concentrations of lysophosphatidylinositol (LPI) were used as shown in the closed circles. These data indicated that a robust agonist signal was observed at 10 μM LPI. Based on these findings, 16 μM LPI was incubated with various concentrations of KLS-13019 to test for antagonistic properties (closed inverted triangles). These data indicated that at 30 μM, KLS-13019 completely blocked LPI-mediated increases in the β-arrestin signal back to control levels. Incubation of KLS-13019 alone produced no detectible increases in β-arrestin signal, indicating that no agonist activity of KLS-13019 was apparent in this human GPR55 system. All assays were conducted with an N=10. Together, these data support the conclusion that KLS-13019 exhibited an antagonist action against LPI when applied in a molar concentration 1.9-fold greater than LPI in the DiscoverX human GPR 55 system.

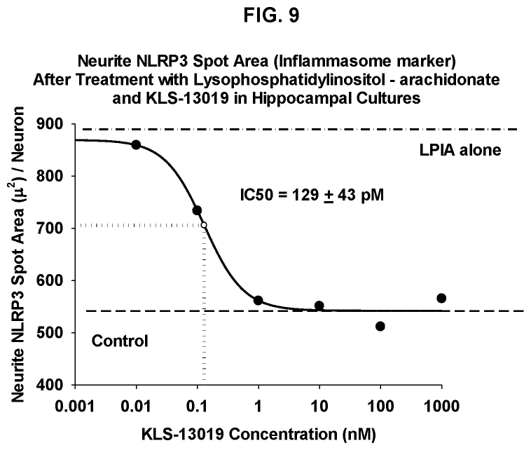

To determine the EC50 of responses of KLS-13019 treatment on the NLRP3 inflammasome, the spot areas of neuritic NLRP3 were measured in cultured hippocampal neurons utilizing a reversing paradigm, with 1 nM LPIA producing the inflammatory signal. The intent of these studies was to extend the scope of treatment of KLS-13019 to anti-inflammatory actions of the central nervous system with the utilization of dissociated hippocampal cultures and LPIA as the inflammatory agonist that interacted with the proinflammatory GPR55 receptor. Utilizing a similar reversal paradigm as that employed with DRG, hippocampal cultures were pre-treated for 4 hours with 1 nM LPIA to produce an inflammatory response as measured with NLRP3 IR spot area. The results of this pre-treatment are shown in FIG. 9 as the reference line labeled LPIA only (dash-dot-dash). After the pre-treatment, various concentrations of KLS-13019 were added to the cultures, with the LPIA not removed from the culture medium. After an additional 4-hour treatment, the cultures were assayed by high content fluorescent imaging to measure changes in the spot area of NLRP3 in neurites. Treatment with KLS-13019 produced a concentration-dependent decrease in neuritic NLRP3 area that exhibited an IC50 of 129±43 pM. Full efficacy of the reversal was apparent in that treatment with 1 nM KLS -13019 produced NLRP3 spot areas that were not different from that of control cultures. The reference line of the control cultures at the end of the experiment are shown as a medium dashed line. These data indicate that KLS-13019 treatment with an equimolar ratio to that of LPIA produced full efficacy reversal of the increased NLRP3 spot area.

While the invention has been described in detail and with reference to specific examples thereof, it will be apparent to one skilled in the art that various changes and modifications can be made therein without departing from the spirit and scope thereof.

### REFERENCES

1. Bonezzi et al. (1999). Treatment options in postherpetic neuralgia. Acta Neurologica Scandinavica, 100, 25-35.
2. Brenneman et al. (2018). Pharmacological comparisons between cannabidiol and KLS-13019. Journal of Molecular Neuroscience, 66(1), 121-134.
3. Brenneman et al. (2019). Knockdown siRNA targeting the mitochondrial sodium-calcium exchanger-1 inhibits the protective effects of two cannabinoids against acute paclitaxel toxicity. Journal of Molecular Neuroscience, 68(4), 603-619.
4. Duggett et al. (2016). Oxidative stress in the development, maintenance and resolution of paclitaxel-induced painful neuropathy. Neuroscience, 333, 13-26.
5. Gangadharan et al. (2013). A novel biological role for the phospholipid lysophosphatidylinositol in nociceptive sensitization via activation of diverse G-protein signalling pathways in sensory nerves in vivo. Pain, 154(12), 2801-2812.
6. Guerrero-Alba et al. (2019). Some prospective alternatives for treating pain: the endocannabinoid system and its putative receptors GPR18 and GPR55. Frontiers in pharmacology, 9, 1496.
7. Gutierrez-Gutierrez et al. (2010). Chemotherapy-induced peripheral neuropathy: clinical features, diagnosis, prevention and treatment strategies. Clinical and Translational Oncology, 12(2), 81-91.
8. Han et al. (2013). Pathobiology of cancer chemotherapy-induced peripheral neuropathy (CIPN). Frontiers in pharmacology, 4, 156.
9. Ji et al. (2018). Neuroinflammation and central sensitization in chronic and widespread pain. Anesthesiology: The Journal of the American Society of Anesthesiologists, 129(2), 343-366.
10. Krames (2014). The role of the dorsal root ganglion in the development of neuropathic pain. Pain Medicine, 15(10), 1669-1685.
11. Kinney et al. (2016). Discovery of KLS-13019, a cannabidiol-derived neuroprotective agent, with improved potency, safety, and permeability. ACS Medicinal Chemistry Letters, 7(4), 424-428.
12. Matsuda et al. (2019). Roles of inflammation, neurogenic inflammation, and neuroinflammation in pain. Journal of anesthesia, 33(1), 131-139.
13. Zeng et al. (2019). Paclitaxel enhances the innate immunity by promoting NLRP3 inflammasome activation in macrophages. Frontiers in immunology, 10, 72.
14. U.S. Pat. No. 9,611,213 B2
15. U.S. Pat. No. 10,004,722 B2

1. A method of treating or preventing at least one of inflammation and pain in a subject, said method comprising administering to the subject an effective amount of a functionalized 1,3-benzene diol represented by a formula selected from the group consisting of:
(a) formula (I): 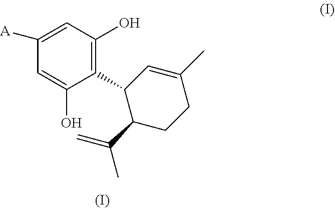
including hydrates, solvates, pharmaceutically acceptable salts, prodrugs and complexes thereof, wherein:
A is selected from the group consisting of 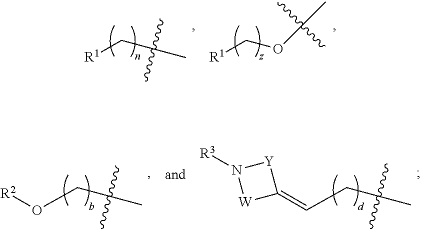
z is 0, 1, or 2;
when A is 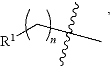
R¹ is selected from the group consisting of 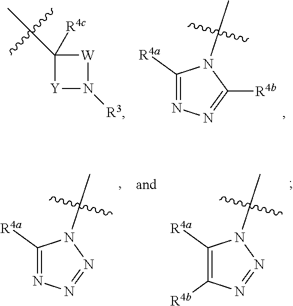
when A is 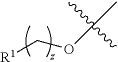
and z is 0, R¹ is 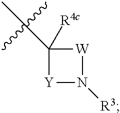
when A is 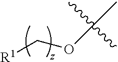
and z is 1, R¹ is 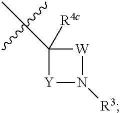
when A is 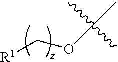
and z is 2, R¹ is selected from the group consisting of 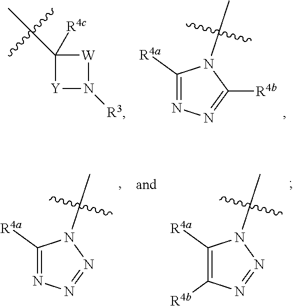
when R¹ is 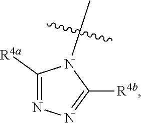
n is not 0;
when R¹ 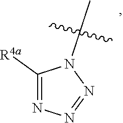
n is not 0;
when R¹ is 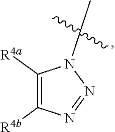
n is not 0;
R² is 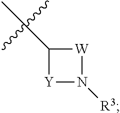
W is (CH₂)ₘ;
m is 1 or 2;
Y is (CH₂)_{q};
q is 1 or 2;
n is 0, 1, 2, or 3;
b is 0, 1, 2, or 3;
d is 0, 1, 2, or 3;
R³ is selected from the group consisting of COR⁵, CO₂R⁶, CONR^{7a}R^{7b}, SO²NR^{7a}R^{7b}, SO₂R⁸, and optionally substituted heteroaryl;
R^{4a} and R^{4b} are each independently selected from the group consisting of hydrogen and C₁₋₆ alkyl;
R^{4c} is selected from the group consisting of hydrogen and OH;
R⁵ is selected from the group consisting of hydrogen, unsubstituted C₁₋₆ alkyl, substituted C₁₋₆ alkyl, unsubstituted heteroaryl, substituted heteroaryl, —C(R^{9a}R^{9b})NR^{7a}R^{7b}, and —C(R^{9a}R^{9b})OR¹⁰;
R⁶ is unsubstituted C₁₋₆ alkyl or substituted C₁₋₆ alkyl;
R^{7a} and R^{7b} are each independently selected from the group consisting of hydrogen, unsubstituted C₁₋₆ alkyl and substituted C₁₋₆ alkyl;
R⁸ is selected from the group consisting of hydrogen, unsubstituted C¹⁻⁶ alkyl, substituted C₁₋₆ alkyl, unsubstituted heteroaryl and substituted heteroaryl;
R^{9a} and R^{9b} are each independently selected from the group consisting of hydrogen, C₁₋₆ alkyl, C₃₋₇ branched alkyl, CH₂OH, CH(OH)CH₃, CH₂Ph, CH₂(4-OH-Ph), (CH₂)₄NH₂, (CH₂)₃NHC(NH₂)NH, CH₂(3-indole), CH₂(5-imidazole), CH₂CO₂H, CH₂CH₂CO₂H, CH₂CONH₂, and CH₂CH₂CONH₂; and
R¹⁰ is selected from the group consisting of hydrogen and C₁₋₆ alkyl;
(b) formula (II): 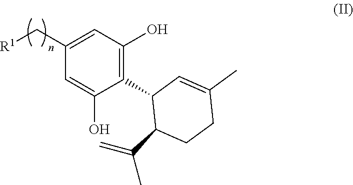
including hydrates, solvates, pharmaceutically acceptable salts, and complexes thereof, wherein R¹ and n of formula (II) are as defined above with respect to formula (I);
(c) formula (III): 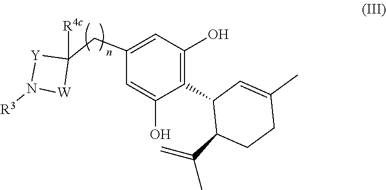
including hydrates, solvates, pharmaceutically acceptable salts, and complexes thereof, wherein R³, R^{4c}, Y, W, and n of formula (III) are as defined above with respect to formula (I);
(d) formula (IV): 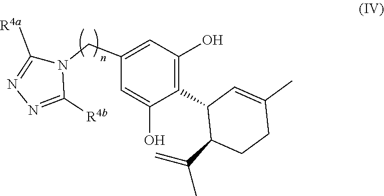
including hydrates, solvates, pharmaceutically acceptable salts, and complexes thereof, wherein n, R^{4a} and R^{4b} are as defined above with respect to formula (I);
(e) formula (V): 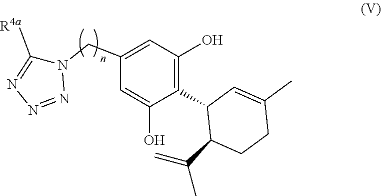
including hydrates, solvates, pharmaceutically acceptable salts, and complexes thereof, wherein n and R^{4a} are as defined above with respect to formula (I);
(f) formula (VI): 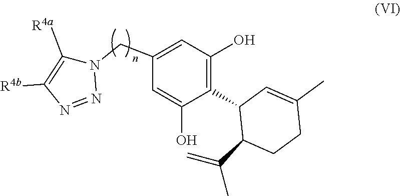
including hydrates, solvates, pharmaceutically acceptable salts, and complexes thereof, wherein n, R^{4a} and R^{4b} are as defined above with respect to formula (I);
(g) formula (VII): 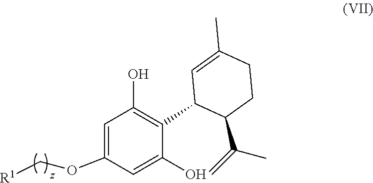
including hydrates, solvates, pharmaceutically acceptable salts, and complexes thereof, wherein R¹ and z are as defined above with respect to formula (I);
(h) formula (VIII): 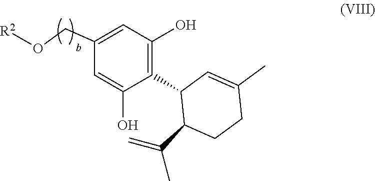
including hydrates, solvates, pharmaceutically acceptable salts, and complexes thereof, wherein R² and b are as defined above with respect to formula (I);
(i) formula (IX): 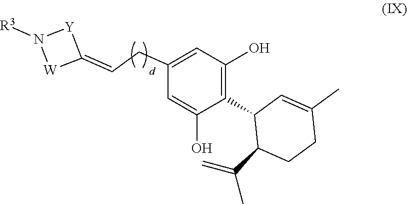
including hydrates, solvates, pharmaceutically acceptable salts, and complexes thereof, wherein R³, Y, W, and d of formula (IX) are as defined above with respect to formula (I); and
(j) formula (X): 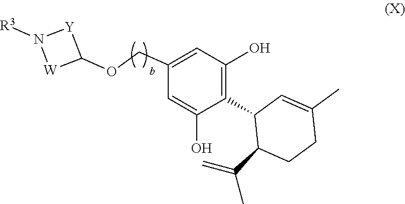
including hydrates, solvates, pharmaceutically acceptable salts, and complexes thereof, wherein R³, Y, W, and b of formula (X) are as defined above with respect to formula (I).

2. The method of claim 1, wherein the functionalized 1,3-benzene diol is represented by the following formula:

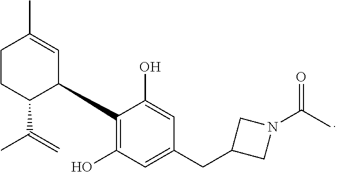 [SMILES: C=C(C)[C@@H]1CCC(C)=C[C@H]1c1c(O)cc(CC2CN(C(C)=O)C2)cc1O]

3. The method of claim 1, wherein the inflammation and pain are associated with a disorder in which GPR55 receptor is expressed in a target tissue of the subject and the functionalized 1,3-benzene diol is a GPR55 antagonist.

4. The method of claim 1, wherein the inflammation and pain are associated with a disorder selected from the group consisting of a chemotherapy-induced peripheral neuropathy, a traumatic brain injury, a traumatic spinal cord injury, a stroke, an autoimmune disease, a viral infection, a surgical trauma, osteoarthritis and chronic opioid treatment.

5. The method of claim 1, wherein the inflammation and pain are associated with a chemotherapy-induced peripheral neuropathy and the functionalized 1,3-benzene diol is administered before, during and/or after administering to the subject a chemotherapeutically effective amount of at least one chemotherapeutic agent selected from the group consisting of vincristine, vinblastine, paclitaxel, docetaxel, bortezomib, cisplatin, oxaliplatin and carboplatin.

6. The method of claim 5, wherein the functionalized 1,3-benzene diol is effective to decrease levels of circulating or tissue cytokines produced in response to the administering of the at least one chemotherapeutic agent.

7. The method of claim 1, wherein the inflammation and pain are associated with chronic opioid treatment, and the functionalized 1,3-benzene diol decreases CNS inflammation associated with chronic opioid use and/or reduces or eliminates opioid dependency.

8. The method of claim 1, wherein the inflammation and pain are associated with diabetic neuropathy.

9. The method of claim 1, wherein the inflammation and pain are associated with post-traumatic stress disorder.

10. The method of claim 1, wherein the inflammation and pain are associated with a neurodegenerative disease that has an oxidative stress component.

11. The method of claim 1, wherein the subject is a human.

12. A chemotherapeutic method, comprising:
administering to a patient a chemotherapeutically effective amount of at least one chemotherapeutic agent selected from the group consisting of vincristine, vinblastine, paclitaxel, docetaxel, bortezomib, cisplatin, oxaliplatin and carboplatin; and
administering to the patient at least one functionalized 1,3-benzene diol in an amount effective to treat or prevent at least one of inflammation and pain associated with administering the at least one chemotherapeutic agent to the patient, wherein the at least one functionalized 1,3-benzene diol is represented by a formula selected from the group consisting of:
(a) formula (I): 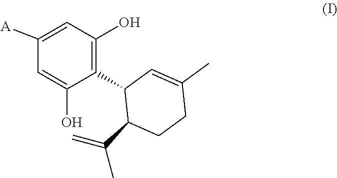
including hydrates, solvates, pharmaceutically acceptable salts, prodrugs and complexes thereof, wherein:
A is selected from the group consisting of 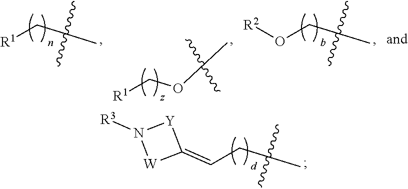
z is 0, 1, or 2;
when A is 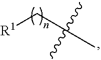
R¹ is selected from the group consisting of 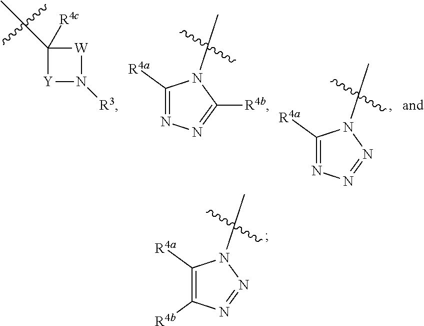
when A is 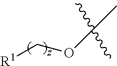
and z is 0, R¹ is 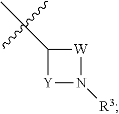
when A is 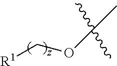
and z is 1, R¹ is 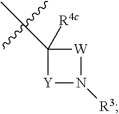
when A is 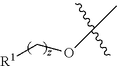
and z is 2, R¹ is selected from the group consisting of 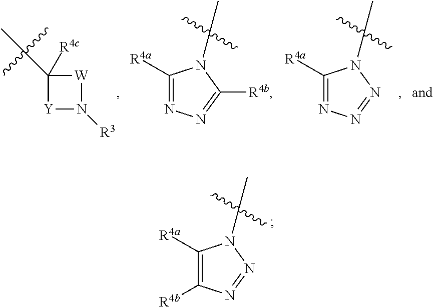
when R¹ is 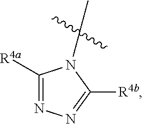
n is not 0;
when R¹ is 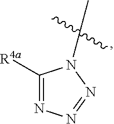
n is not 0;
when R¹ is 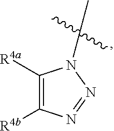
n is not 0;
R² is 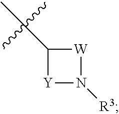
W is (CH₂)ₘ;
m is 1 or 2;
Y is (CH₂)_{q};
q is 1 or 2;
n is 0, 1, 2, or 3;
b is 0, 1, 2, or 3;
d is 0, 1, 2, or 3;
R³ is selected from the group consisting of COR⁵, CO₂R⁶, CONR^{7a}R^{7b}, SO₂NR^{7a}R^{7b}, SO₂R⁸, and optionally substituted heteroaryl;
R^{4a} and R^{4b} are each independently selected from the group consisting of hydrogen and C₁₋₆ alkyl;
R^{4c} is selected from the group consisting of hydrogen and OH;
R⁵ is selected from the group consisting of hydrogen, unsubstituted C₁₋₆ alkyl, substituted C₁₋₆ alkyl, unsubstituted heteroaryl, substituted heteroaryl, —C(R^{9a}R^{9b})NR^{7a}R^{7b}, and —C(R^{9a}R^{9b})OR¹⁰;
R⁶ is unsubstituted C₁₋₆ alkyl or substituted C₁₋₆ alkyl;
R^{7a} and R^{7b} are each independently selected from the group consisting of hydrogen, unsubstituted C₁₋₆ alkyl and substituted C₁₋₆ alkyl;
R⁸ is selected from the group consisting of hydrogen, unsubstituted C₁₋₆ alkyl, substituted C₁₋₆ alkyl, unsubstituted heteroaryl and substituted heteroaryl;
R^{9a} and R^{9b} are each independently selected from the group consisting of hydrogen, C₁₋₆ alkyl, C₃₋₇ branched alkyl, CH₂OH, CH(OH)CH₃, CH₂Ph, CH₂(4-OH-Ph), (CH₂)₄NH₂, (CH₂)₃NHC(NH₂)NH, CH₂(3-indole), CH₂(5-imidazole), CH₂CO₂H, CH₂CH₂CO₂H, CH₂CONH₂, and CH₂CH₂CONH₂; and
R¹⁰ is selected from the group consisting of hydrogen and C₁₋₆ alkyl;
(b) formula (II): 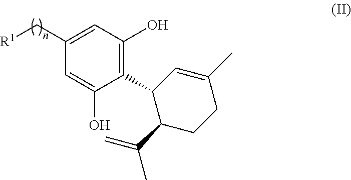
including hydrates, solvates, pharmaceutically acceptable salts, and complexes thereof, wherein R¹ and n of formula (II) are as defined above with respect to formula (I);
(c) formula (III): 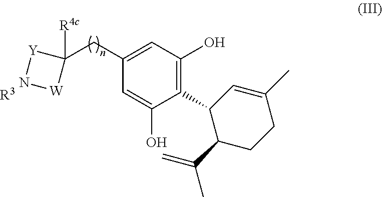
including hydrates, solvates, pharmaceutically acceptable salts, and complexes thereof, wherein R³, R^{4c}, Y, W, and n of formula (III) are as defined above with respect to formula (I);
(d) formula (IV): 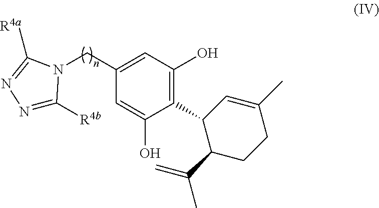
including hydrates, solvates, pharmaceutically acceptable salts, and complexes thereof, wherein n, R^{4a} and R^{4b} are as defined above with respect to formula (I);
(e) formula (V): 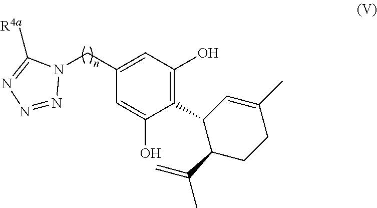
including hydrates, solvates, pharmaceutically acceptable salts, and complexes thereof, wherein n and R^{4a} are as defined above with respect to formula (I);
(f) formula (VI): 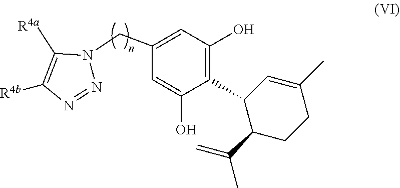
including hydrates, solvates, pharmaceutically acceptable salts, and complexes thereof, wherein n, R^{4a} and R^{4b} are as defined above with respect to formula (I);
(g) formula (VII): 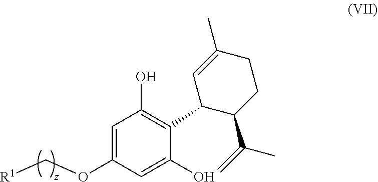
including hydrates, solvates, pharmaceutically acceptable salts, and complexes thereof, wherein R¹ and z are as defined above with respect to formula (I);
(h) formula (VIII): 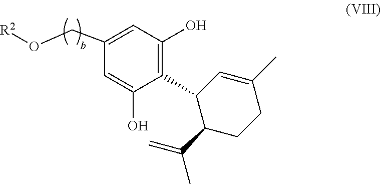
including hydrates, solvates, pharmaceutically acceptable salts, and complexes thereof, wherein R² and b are as defined above with respect to formula (I);
formula (IX): 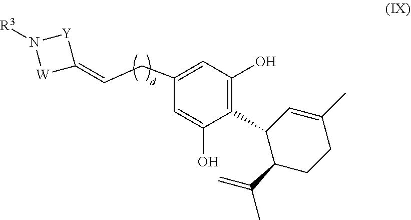
including hydrates, solvates, pharmaceutically acceptable salts, and complexes thereof, wherein R³, Y, W, and d of formula (IX) are as defined above with respect to formula (I); and
(j) formula (X): 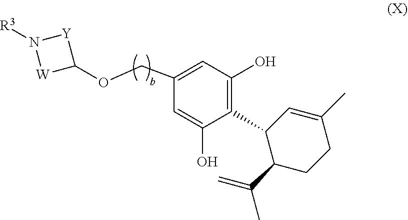
including hydrates, solvates, pharmaceutically acceptable salts, and complexes thereof, wherein R³, Y, W, and b of formula (X) are as defined above with respect to formula (I).

13. The method of claim 12, wherein the at least one functionalized 1,3-benzene diol is represented by the following formula:

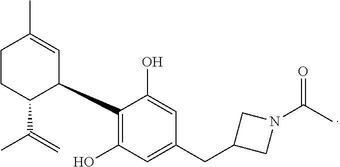 [SMILES: C=C(C)[C@@H]1CCC(C)=C[C@H]1c1c(O)cc(CC2CN(C(C)=O)C2)cc1O]

14. The method of claim 12, wherein the at least one functionalized 1,3-benzene diol and the at least one chemotherapeutic agent are administered together.

15. A chemotherapeutic composition, comprising:
at least one chemotherapeutic agent selected from the group consisting of vincristine, vinblastine, paclitaxel, docetaxel, bortezomib, cisplatin, oxaliplatin and carboplatin; and
at least one functionalized 1,3-benzene diol represented by a formula selected from the group consisting of:
(a) formula (I): 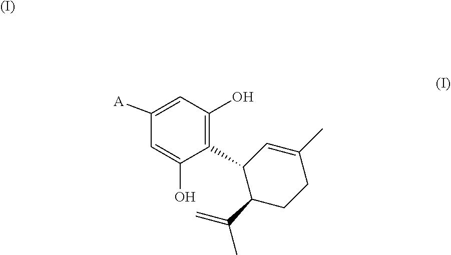
including hydrates, solvates, pharmaceutically acceptable salts, prodrugs and complexes thereof, wherein:
A is selected from the group consisting of 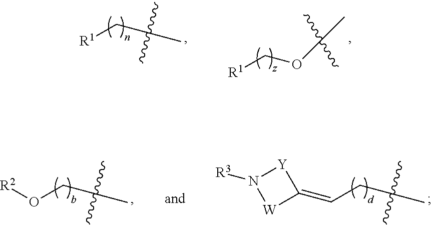
z is 0, 1, or 2;
when A is 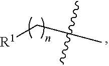
R¹ is selected from the group consisting of 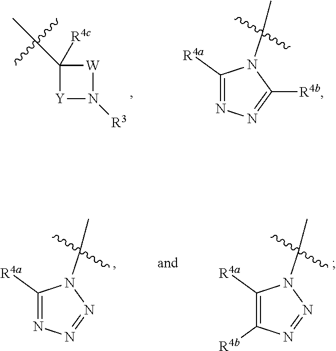
when A is 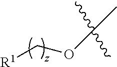
and z is 0, R¹ is 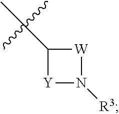
when A is 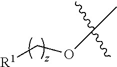
and z is 1, R¹ is 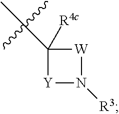
when A is 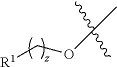
and z is 2, R¹ is selected from the group consisting of 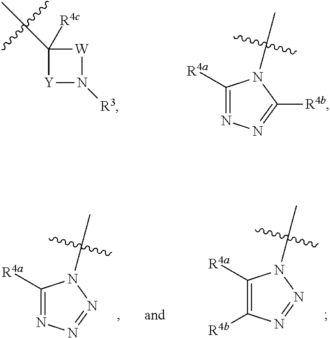
when R¹ is 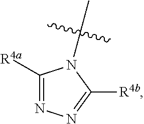
n is not 0;
when R¹ is 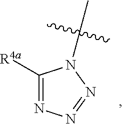
n is not 0;
when R¹ is 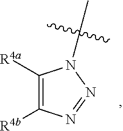
n is not 0;
R² is 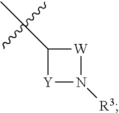
W is (CH₂)ₘ;
m is 1 or 2;
Y is (CH₂)_{q};
q is 1 or 2;
n is 0, 1, 2, or 3;
b is 0, 1, 2, or 3;
d is 0, 1, 2, or 3;
R³ is selected from the group consisting of COR⁵, CO₂R⁶, CONR^{7a}R^{7b}, SO₂NR^{7a}R^{7b}, SO₂R⁸, and optionally substituted heteroaryl;
R^{4a} and R^{4b} are each independently selected from the group consisting of hydrogen and C₁₋₆ alkyl;
R^{4c} is selected from the group consisting of hydrogen and OH;
R⁵ is selected from the group consisting of hydrogen, unsubstituted C₁₋₆ alkyl, substituted C₁₋₆ alkyl, unsubstituted heteroaryl, substituted heteroaryl, —C(R^{9a}R^{9b})NR^{7a}R^{7b}, and —C(R^{9a}R^{9b})OR¹⁰;
R⁶ is unsubstituted C₁₋₆ alkyl or substituted C₁₋₆ alkyl;
R^{7a} and R^{7b} are each independently selected from the group consisting of hydrogen, unsubstituted C₁₋₆ alkyl and substituted C₁₋₆ alkyl;
R⁸ is selected from the group consisting of hydrogen, unsubstituted C₁₋₆ alkyl, substituted C₁₋₆ alkyl, unsubstituted heteroaryl and substituted heteroaryl;
R^{9a} and R^{9b} are each independently selected from the group consisting of hydrogen, C₁₋₆ alkyl, C₃₋₇ branched alkyl, CH₂OH, CH(OH)CH₃, CH₂Ph, CH₂(4-OH-Ph), (CH₂)₄NH₂, (CH₂)₃NHC(NH₂)NH, CH₂(3-indole), CH₂(5-imidazole), CH₂CO₂H, CH₂CH₂CO₂H, CH₂CONH₂, and CH₂CH₂CONH₂; and
R¹⁰ is selected from the group consisting of hydrogen and C₁₋₆ alkyl;
(b) formula (II): 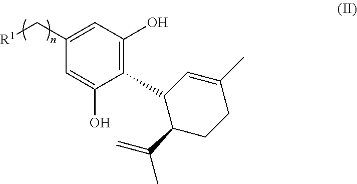
including hydrates, solvates, pharmaceutically acceptable salts, and complexes thereof, wherein R¹ and n of formula (II) are as defined above with respect to formula (I);
(c) formula (III): 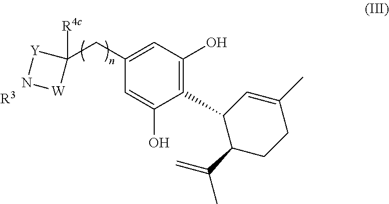
including hydrates, solvates, pharmaceutically acceptable salts, and complexes thereof, wherein R³, R^{4c}, Y, W, and n of formula (III) are as defined above with respect to formula (I);
(d) formula (IV): 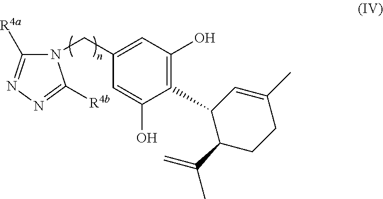
including hydrates, solvates, pharmaceutically acceptable salts, and complexes thereof, wherein n, R^{4a} and R^{4b} are as defined above with respect to formula (I);
(e) formula (V): 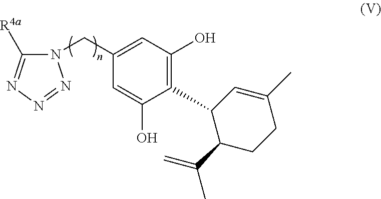
including hydrates, solvates, pharmaceutically acceptable salts, and complexes thereof, wherein n and R^{4a} are as defined above with respect to formula (I);
(f) formula (VI): 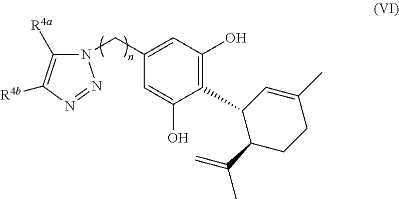
including hydrates, solvates, pharmaceutically acceptable salts, and complexes thereof, wherein n, R^{4a} and R^{4b} are as defined above with respect to formula (I);
(g) formula (VII): 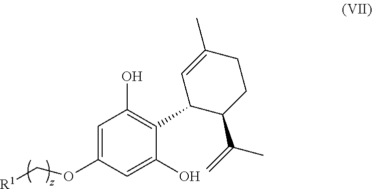
including hydrates, solvates, pharmaceutically acceptable salts, and complexes thereof, wherein R¹ and z are as defined above with respect to formula (I);
(h) formula (VIII): 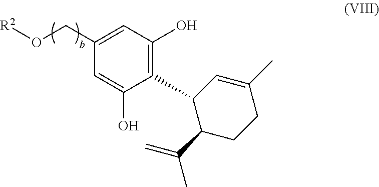
including hydrates, solvates, pharmaceutically acceptable salts, and complexes thereof, wherein R² and b are as defined above with respect to formula (I);
(i) formula (IX): 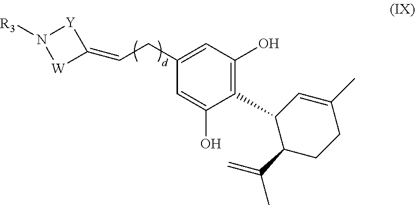
including hydrates, solvates, pharmaceutically acceptable salts, and complexes thereof, wherein R³, Y, W, and d of formula (IX) are as defined above with respect to formula (I); and
(j) formula (X): 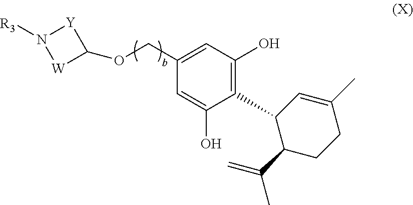
including hydrates, solvates, pharmaceutically acceptable salts, and complexes thereof, wherein R³, Y, W, and b of formula (X) are as defined above with respect to formula (I).

16. The chemotherapeutic composition of claim 15, wherein the at least one chemotherapeutic agent is provided in a chemotherapeutically effective amount and the at least one functionalized 1,3-benzene diol is provided in an amount effective to treat or prevent at least one of inflammation and pain associated with administering the at least one chemotherapeutic agent to the patient.

17. The chemotherapeutic composition of claim 15, wherein the at least one functionalized 1,3-benzene diol is represented by the following formula:

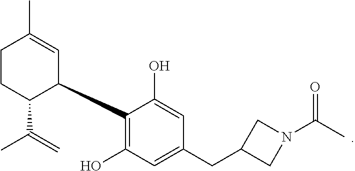 [SMILES: C=C(C)[C@@H]1CCC(C)=C[C@H]1c1c(O)cc(CC2CN(C(C)=O)C2)cc1O]

18. The chemotherapeutic composition of claim 17, wherein the at least one chemotherapeutic agent is provided in a chemotherapeutically effective amount and the at least one functionalized 1,3-benzene diol is provided in an amount effective to treat or prevent at least one of inflammation and pain associated with administering the at least one chemotherapeutic agent to the patient.
